(19) United States
(12) Patent Application Publication
(10) Pub. No.: US 2025/0025542 A1
(43) Pub. Date: Jan. 23, 2025

(54) COMPOSITIONS AND METHODS FOR INDUCING IMMUNE TOLERANCE

(71) Applicant: THE UNIVERSITY OF CHICAGO, Chicago, IL (US)

(72) Inventors: Aaron ESSER-KAHN, Chicago, IL (US); Peter DEAK, Chicago, IL (US)

(73) Assignee: THE UNIVERSITY OF CHICAGO, Chicago, IL (US)

(21) Appl. No.: 18/708,916

(22) PCT Filed: Nov. 9, 2022
(86) PCT No.: PCT/US2022/079522

§ 371 Date: May 9, 2024

Related U.S. Application Data
(60) Provisional application No. 63/277,579, filed on Nov. 9, 2021.

(51) Int. Cl. A61K 39/00; A61K 9/127; A61K 31/365; A61K 31/366; A61K 31/381; A61K 31/437; A61K 31/444; A61K 31/496; A61K 31/519; A61K 31/573; A61K 31/575; A61P 37/06

(52) U.S. Cl. CPC A61K 39/001; A61K 9/127; A61K 31/365; A61K 31/366; A61K 31/381; A61K 31/437; A61K 31/444; A61K 31/496; A61K 31/519; A61K 31/573; A61K 31/575; A61K 39/0008; A61P 37/06

(57) ABSTRACT

Aspects of the present disclosure are directed to methods and compositions for generation of antigen-specific regulatory T cells. Certain aspects relate to methods and compositions for generating tolerogenic antigen presenting cells, including tolerogenic dendritic cells. The present disclosure includes compositions, including nanocarrier compositions, comprising TLR agonists, and immunosuppressive agents and optionally an antigen. Also disclosed are methods for use of such compositions in generation of tolerogenic antigen presenting cells and treatment of certain conditions, including autoimmune and inflammatory conditions.

### CROSS-REFERENCE FOR RELATED APPLICATIONS

This application claims the benefit of priority of U.S. Provisional Patent Application No. 63/277,579 filed Nov. 9, 2021, which is hereby incorporated by reference in its entirety.

### STATEMENT OF GOVERNMENT SUPPORT

This invention was made with government support under grant numbers AI124286 and AI147517 awarded by the National Institutes of Health. The government has certain rights in the invention.

### BACKGROUND

### I. Field of the Invention

This invention relates to at least the fields of immunology and medicine.

### II. Background

Autoimmune diseases are a growing problem in the western world and are particularly difficult to treat due to the wide range of disease pathogenesis and symptoms. As a result, treatments typically include broad immune suppression that leaves patients vulnerable for other infections. While many new autoimmune therapies have been developed in an attempt to generate autoantigen specific immune suppression, many challenges remain to generate active and antigen specific T regulatory cells (Tregs). Tregs are critical for the maintenance of long lasting tolerance for autoimmune patients, as they actively suppress autoreactive T and B cells and promote their anergy and deletion, while sparing other essential immune cells. As a result, there is a need for methods and compositions capable of generation of long lived and active autoantigen-specific Tregs.

### SUMMARY

The present disclosure fulfills certain needs in the fields of immunology and medicine by providing compositions and methods for generation of tolerogenic antigen presenting cells and antigen-specific T regulatory cells. The present disclosure includes compositions (described in some cases as “tolerogenic compositions”) comprising an immune activator (e.g., TLR agonist) and one or more immunosuppressive agents, in some cases also comprising one or more antigens, which components may be encapsulated in and/or attached to a nanocarrier such as a liposome. Also disclosed are methods for treating a subject for cancer or for an autoimmune condition comprising administering one or more tolerogenic compositions to the subject. Methods for generating tolerogenic dendritic cells are also disclosed, including in vitro, ex vivo, and in vivo methods, such methods comprising providing one or more tolerogenic compositions described herein to a population of dendritic cells.

The disclosure also describes methods for generating tolerogenic dendritic cells, methods for generating T regulatory cells, methods for activating T regulatory cells, methods for treating an autoimmune condition, methods for treating an inflammatory condition, methods for treating or preventing graft versus host disease, methods for treating or preventing graft rejection, methods for suppressing an immune response, methods for treating multiple sclerosis, and methods for treating diabetes, as well as nanocarriers, liposomes, immunosuppressive agents, tolerogenic compositions, and pharmaceutical compositions. Compositions of the disclosure may comprise at least 1, 2, 3, 4, 5, 6, 7, 8, 9, 10, or more of the following components: a nanocarrier, a liposome, an antigen, an antigenic peptide, a multiple sclerosis antigen, an immune activator, a TLR agonist, flagellin, LPS, Pam2CSK4, R848, a CpG oligonucleotide, an immunosuppressive agent, cucurbitacin I, costunolide, MLN120B, parthenolide, peficitinib, oclacitinib maleate, AD80, cucurbitacin B, CEP-33779, dehydrocostus lactone, dexamethasone, simvastatin, and SC 514. Any one or more of the preceding components may be specifically excluded from aspects of the disclosure. Methods of the disclosure may comprise at least 1, 2, 3, or more of the following steps: administering an antigen to a subject, administering a TLR agonist to a subject, administering an immunosuppressive agent to a subject, administering a nanocarrier to a subject, administering a tolerogenic composition to a subject, sequentially administering two separate tolerogenic compositions to a subject, administering dexamethasone to a subject, administering simvastatin to a subject, administering SC 514 to a subject, simultaneously administering dexamethasone, simvastatin, and SC 514 to a subject, diagnosing a subject as having an autoimmune condition, diagnosing a subject as having an inflammatory condition, administering a transplant to a subject, obtaining immune cells from a subject, obtaining dendritic cells from a subject, obtaining dendritic cell precursors from a subject, providing a TLR agonist to a population of immune cells, and providing an immunosuppressive agent to a population of immune cells. Any one or more of the preceding steps may be specifically excluded from aspects of the disclosure.

Disclosed herein is a composition comprising (a) a TLR agonist and (b) one or more immunosuppressive agents. The composition may also comprise an antigen. Compositions of the disclosure, such as pharmaceutical compositions may comprise a pharmaceutical composition comprising (i) a nanocarrier disclosed herein and (ii) a pharmaceutically acceptable excipient. Further disclosed is a method for treating a subject for an autoimmune or inflammatory condition, the method comprising administering to the subject an effective amount of a composition or nanocarrier described herein.

Also disclosed is a method for treating a subject for an autoimmune or inflammatory condition, the method comprising administering to the subject an effective amount of: (i) a TLR agonist; and (ii) one or more immunosuppressive agents. The method may comprise or further comprise administering an antigen associated with the autoimmune or inflammatory condition. The method may comprise or further comprise administering to the subject an effective amount of: (1) an additional or second TLR agonist; and (2) one or more additional or second immunosuppressive agents. The method may comprise or further comprise administration of an additional or second antigen associated with the autoimmune or inflammatory condition. The first administered TLR agonist, first immunosuppressive agent, and optional first antigen and the additional or second administered TLR agonist, second immunosuppressive agent, and optional second antigen may be administered to the subject simultaneously. The first administered TLR agonist, first immunosuppressive agent, and optional first antigen and the additional or second administered TLR agonist, second immunosuppressive agent, and optional second antigen may be administered to the subject sequentially. The additional or second administered TLR agonist, second immunosuppressive agent, and optional second antigen may be administered to the subject subsequent to administering the first TLR agonist, first immunosuppressive agent, and optional first antigen. The additional or second administered TLR agonist, second immunosuppressive agent, and optional second antigen may be administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 40, 44, 48, or 72 hours, or more, after administering the first TLR agonist, first immunosuppressive agent, and optional first antigen to the subject. The additional or second administered TLR agonist, second immunosuppressive agent, and optional second antigen may be administered to the subject at least 12 hours after administering the first TLR agonist, first immunosuppressive agent, and optional first antigen to the subject. The additional or second administered TLR agonist, second immunosuppressive agent, and optional second antigen may be administered to the subject at least 18 hours after administering the first TLR agonist, first immunosuppressive agent, and optional first antigen to the subject. The additional or second administered TLR agonist, second immunosuppressive agent, and optional second antigen may be administered to the subject at least 24 hours after administering the first TLR agonist, first immunosuppressive agent, and optional first antigen to the subject. The additional or second administered TLR agonist, second immunosuppressive agent, and optional second antigen may be administered to the subject prior to administering the first TLR agonist, first immunosuppressive agent, and optional first antigen to the subject.

Disclosed herein is a method for generating tolerogenic dendritic cells, the method comprising administering, to a population of dendritic cells or dendritic cell precursors: (a) one or more TLR agonists; and (b) one or more immunosuppressive agents. The population of dendritic cells or dendritic cell precursors may be incubated with the TLR agonist(s) and immunosuppressive agent(s) for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 40, 44, 48, or 72 hours, or more. The population of dendritic cells or dendritic cell precursors may be incubated with the TLR agonist(s) and immunosuppressive agent(s) for at least 12 hours. The population of dendritic cells or dendritic cell precursors may be incubated with the TLR agonist(s) and immunosuppressive agent(s) for at least 18 hours. The population of dendritic cells or dendritic cell precursors may be incubated with the TLR agonist(s) and immunosuppressive agent(s) for at least 24 hours. The methods may comprise further providing an antigen to the population of dendritic cells or dendritic cell precursors. Methods may comprise or further comprise administering to the population of dendritic cells or dendritic cell precursors: (a) one or more additional TLR agonists; and (b) one or more additional immunosuppressive agents. The method may be performed ex vivo. The additional TLR agonist(s) and immunosuppressive agent(s) may be provided at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 40, 44, 48, or 72 hours, or more, after providing the first TLR agonist(s) and immunosuppressive agent(s). The additional TLR agonist(s) and immunosuppressive agent(s) may be provided at least 12 hours after administering the first TLR agonist(s) and immunosuppressive agent(s) to the population of dendritic cells or dendritic cell precursors. The additional TLR agonist(s) and immunosuppressive agent(s) may be provided at least 18 hours after administering the first TLR agonist(s) and immunosuppressive agent(s) to the population of dendritic cells or dendritic cell precursors. The additional TLR agonist(s) and immunosuppressive agent(s) may be provided at least 24 hours after administering the first TLR agonist(s) and immunosuppressive agent(s) to the population of dendritic cells or dendritic cell precursors.

The one or more immunosuppressive agents comprise comprise one or more of cucurbitacin I, costunolide, MVLN120B, parthenolide, peficitinib, oclacitinib maleate, AD80, cucurbitacin B, CEP-33779, Dehydrocostus Lactone, dexamethasone, simvastatin, SC 514, ly294002, minocycline, hydroxychloroquine, Sialyl Lewis X, Thymic Stromal Lymphopoietin (TSLP), and lifitegrast. The one or more immunosuppressive agents may comprise one or more of dexamethasone, simvastatin, and SC 514. The one or more immunosuppressive agents may comprise two or more of dexamethasone, simvastatin, and SC 514. The one or more immunosuppressive agents may comprise dexamethasone, simvastatin, and SC 514. The dexamethasone, simvastatin, and SC 514 may be at a 1:1:1 concentration ratio.

The TLR agonist may comprise a TLR5 agonist, a TLR9 agonist, a TLR2 agonist, a TLR6 agonist, a TLR2/6 agonist, a TLR7 agonist, a TLR8 agonist, a TLR 7/8 agonist, and/or a TLR4 agonist. The TLR agonist may comprise a TLR5 agonist. The TLR agonist may comprise flagellin. The flagellin may be Bacillus subtilis flagellin. The TLR agonist may comprise a TLR9 agonist. The TLR agonist may comprise a CpG oligonucleotide (CpG ODN). The CpG oligonucleotide may be CpG ODN 1826. The TLR agonist may comprise a TLR2 agonist, a TLR6 agonist. The TLR agonist may comprise a TLR2/6 agonist. The TLR agonist may comprise a TLR7 agonist. The TLR agonist may comprise a TLR8 agonist. The TLR agonist may comprise a TLR 7/8 agonist. The TLR agonist may comprise LPS. The TLR agonist may comprise Pam2CSK4. The TLR agonist may comprise R848. The compositions may comprise, the methods may comprise administration of, or the methods may comprise providing to the population of dendritic cells or precursors a combination of at least one TLR agonist and one immunosuppressive agent. The compositions may comprise, the methods may comprise administration of, or the methods may comprise providing to the population of dendritic cells or precursors a combination of exactly one TLR agonist and exactly one immunosuppressive agent. The immunosuppressive agent and TLR agonist may comprise or consist of cucurbitacin I and LPS. The immunosuppressive agent and TLR agonist may comprise or consist of costunolide and Pam2CSK4. The immunosuppressive agent and TLR agonist may comprise or consist of MLN120B and Pam2CSK4. The immunosuppressive agent and TLR agonist may comprise or consist of parthenolide and Pam2CSK4. The immunosuppressive agent and TLR agonist may comprise or consist of peficitinib and Pam2CSK4. The immunosuppressive agent and TLR agonist may comprise or consist of oclacitinib maleate and R848. The immunosuppressive agent and TLR agonist may comprise or consist of AD80 and Pam2CSK4. The immunosuppressive agent and TLR agonist may comprise or consist of curcurbitacin B and R848. The immunosuppressive agent and TLR agonist may comprise or consist of CEP-33779 and R848. The immunosuppressive agent and TLR agonist may comprise or consist of dehydrocostus lactone and R848.

The molar ratio of TLR agonist to immunosuppressive agent in compositions of the disclosure may be from 1:10-1:100. The molar ratio of TLR agonist to immunosuppressive agent administered to subjects may be from 1:10-1:100. The molar ratio of TLR agonist to immunosuppressive agent in compositions of the disclosure may be from 5:1-20:1. The molar ratio of TLR agonist to immunosuppressive agent administered to subjects may be from 5:1-20:1. The molar or weight ratio of the TLR agonist to immunosuppressive agent administered to the subject or in the composition may be from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 to 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200, or any derivable range therein.

The concentration of the immunosuppressive agent may be 0.1 μM-10 μM. The concentration of the immunosuppressive agent may be, may be at least, or may be at most 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 pM, nM, μM, mM, or any range derivable therein.

The concentration of the TLR agonist may be 1 nM-1 μM. The concentration of the immunosuppressive agent may be, may be at least, or may be at most 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 pM, nM, μM, mM, or any range derivable therein.

The antigen may be a cancer antigen. The antigen may be an antigen associated with an autoimmune or inflammatory condition. The autoimmune or inflammatory condition may be multiple sclerosis. The antigen may be a myelin sheath protein or portion thereof. The myelin sheath protein may be myelin basic protein (MBP), myelin oligodendrocyte glycoprotein (MOG), proteolipid protein (PLP), or myelin associated glycoprotein (MAG). The antigen may be a MOG peptide. The autoimmune or inflammatory condition may be graft versus host disease. The autoimmune or inflammatory condition may be graft rejection. The antigen may be a self-antigen. The antigen may be a foreign antigen.

The disclosure also provides for nanocarriers comprising compositions of the disclosure. In the methods of the disclosure, the TLR agonist, immunosuppressive agent, and optionally an antigen may be in a nanocarrier that is administered to a subject or a population of dendritic cells or precursors. The nanocarrier may be a liposome, wherein the antigen is encapsulated in an interior of the liposome. The nanocarrier may be a liposome, wherein the one or more immunosuppressive agents are encapsulated in a membrane of the liposome. The nanocarrier may be a liposome, a nanoparticle, a dendrimer, or a micelle. The nanocarrier may be a liposome. The TLR agonist may be conjugated to the liposome. The TLR agonist may be displayed on a surface of the liposome.

The TLR agonist, immunosuppressive agent, composition, nanocarrier, and/or antigen may be administered to the subject prior to or together with an allogeneic transplant. The antigen may be an antigen from a graft of the transplant. The method may be for treating or preventing graft versus host disease or graft rejection. The allogeneic transplant may be a bone marrow transplant. The antigen may be a bone marrow antigen. The allogeneic transplant may be an organ transplant. The antigen may be an antigen from the organ.

Disclosed herein is a nanocarrier comprising (a) an antigen associated with an autoimmune condition; (b) a TLR5 agonist or a TLR9 agonist; (c) dexamethasone, (d) simvastatin, and (e) SC 514. Disclosed herein is a liposome comprising (a) an antigen associated with multiple sclerosis; (b) flagellin conjugated to an exterior surface of the liposome; (c) dexamethasone, (d) simvastatin, and (e) SC 514. Disclosed herein is a liposome comprising (a) an antigen associated with multiple sclerosis; (b) a CpG oligonucleotide conjugated to an exterior surface of the liposome; (c) dexamethasone, (d) simvastatin, and (e) SC 514.

Disclosed herein is a method for treating a subject for multiple sclerosis, the method comprising administering to the subject an effective amount of a composition comprising: a liposome comprising: (i) an antigen associated with multiple sclerosis; and (ii) the combination of one of: (a) flagellin, dexamethasone, simvastatin, and SC 514; (b) a CpG oligonucleotide, dexamethasone, simvastatin, and SC 514; (c) cucurbitacin and LPS; (d) costunolide and Pam2CSK4; (e) MLN120B and Pam2CSK4; (f) parthenolide and Pam2CSK4; (g) peficitinib and Pam2CSK4; (h) oclacitinib maleate and R848; (i) AD80 and Pam2CSK4; (j) cucurbitacin B and R848; (k) CEP-33779 and R848; or (1) dehydrocostus lactone and R848.

Disclosed herein is a method for treating or preventing graft versus host disease or graft rejection in a subject, the method comprising administering to the subject an effective amount of a composition comprising: (i) an antigen associated with multiple sclerosis; and (ii) the combination of one of: (a) flagellin, dexamethasone, simvastatin, and SC 514; (b) a CpG oligonucleotide, dexamethasone, simvastatin, and SC 514; (c) cucurbitacin and LPS; (d) costunolide and Pam2CSK4; (e) MLN120B and Pam2CSK4; (f) parthenolide and Pam2CSK4; (g) peficitinib and Pam2CSK4; (h) oclacitinib maleate and R848; (i) AD80 and Pam2CSK4; (j) cucurbitacin B and R848; (k) CEP-33779 and R848; or (1) dehydrocostus lactone and R848.

The TLR agonist, immunosuppressive agent, and optional antigen may be administered to the subject prior to an allogeneic transplant. The TLR agonist, immunosuppressive agent, and optional antigen may be administered together with an allogeneic transplant. The TLR agonist, immunosuppressive agent, and optional antigen may be administered after an allogeneic transplant. The allogeneic transplant may be a bone marrow transplant. The allogeneic transplant may be an organ transplant.

The subject may be a human subject. The subject may be a cow, pig, horse, cat, dog, mammal, non-human primate, rat, mouse, or rabbit.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual, “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also aspects that may be implemented in the context of aspects discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1F. Combining TLR agonist and immunomodulatory drugs generates tolerogenic APCs in vitro. (FIG. 1A) Conceptual diagram representing tolerogenic liposome for generation of T regulatory response—a combination of inhibitors and TLR agonists generate more active Tregs through a push/pull mechanism. (FIG. 1B) Analysis of IL-6 and IL-12 production from combinations of immunomodulatory drugs and TLR agonists. 100 k BMDCs were plated and treated with a combination of 0.1 μg/uLFLA (TLR5 agonist) and immunoinhibitory drugs. After a 14 hr incubation, cells were washed and treated with 0.5 uM CpG. Supernatants were drawn 4 hrs later (for IL-6) and 20 hrs later (for IL-10). Cytokine secretion was measured by ELISA. (FIG. 1C) Analysis of cell surface marker response to combination in BMDCs. Cells were treated with varying concentrations of a 1:1:1 combination of dex, sim, and sc-514 (concentration indicated on x axis is concentration of all inhibitors). Cells were analyzed via flow 24 hrs after CpG addition for PD-L1, CD80 and CD40. (FIGS. 1D-1E) Analysis of tolerogenic response on naïve spleenocytes. Methods from C was repeated on naïve spleenocytes (1 million per sample) in triplicate from three separate mice and then analyzed via flow. CD45+, MHCII+, CD11c+, CD19− cells (DCs) were gated and then stained for (FIG. 1D) PD-L1 and PD-L2 or (FIG. 1E) CD80, CD86 and MHCII. (FIG. 1F) Analysis of TLR agonist/Inhibitor combination lifetime in tolDC phenotypes. BMDCs were plated into 5 different 96 well plates (200 k cells per well) and incubated with 1 uM inhibitor combination (I, 1:1:1 combination of dex:sim:sc-514) in combination with 0.1 μg/mL FLA for 16 hrs. Cells were then washed and treated with 0.5 uM CpG+1 uM I. 20 hrs later, on day 1, one plate of cells was tested for viability using MTT assay and for IL-10 secretion via CBA. The remaining plates were washed and incubated with fresh media. On day 3, one plate of cells was challenged with 0.5 uM CpG for 20 hrs then analyzed for cell viability (line plots) and IL-10 secretion (bar graphs). This procedure was repeated on day 6, 9 and 13 with remaining plates. The TLR agonist and inhibitor treated samples (TLR+I, shown in blue) were compared to cells treated with just inhibitor combination (I, shown in red). Error bars indicate: of SD of biological triplicate experiments.

FIGS. 2A-2H. Lipo^{TLR+I} generate tolDCs that actively uptake antigen in vitro and in vivo. (FIG. 2A) Analysis of Lipo^{TLR+I} In Vitro. 100 k BMDCs were treated with free or liposomal formulations of TLR+I, TLR or I and analyzed via flow cytometry for liposomal uptake. Liposomes were synthesized with AF647-OVA for a total of 1 μg of OVA per 0.1 IM of inhibitor. Free inhibitor formulations were treated with equivalent OVA dose. 1 hour after second treatment, cells were washed and analyzed via flow for OVA internalization (FIG. 2B) In Vivo Analysis of Lipo^{TLR+I} uptake. C57BL/6 mice (4 per group) were injected with either free or Lipo formulations of OVA and combinations of inhibitors to for the following categories (100 μg OVA/mouse, 10 umols inhibitor/mouse, 1 μg FLA/mouse, 10 μg CpG/mouse): (1) OVA alone (PBS), (2) OVA+TLR agonists (TLR), OVA+TLR agonists+dexsamethasone only (TLR+Dex) or OVA+Inhibitor combination (TLR+I). For Lipo formulations, DiD was added at 0.01% total lipid loading to allow fluorescent analysis. Mice were injected with either Lipo or free combinations i.p. with FLA then CpG formulations on consecutive days. 24 hrs after final CpG injection, mice were sacrificed, popliteal and inguinal lymph nodes harvested, disassociated and stained for various immune cell markers. Lymphocytes were analyzed via spectral flow and DC populations (CD45+, MHCII+, CD11c+, CD19−) analyzed for CD40, (FIG. 2C) CD80 (FIG. 2D) CD86 (FIG. 2E) CD103 (FIG. 2F) PD-L1 (FIG. 2G) PD-L2. (FIG. 2H) Mice treated with liposomes were gated on Liposome+ and PD-L1/2+ cell populations. Error bars indicate ± of SD of each mouse group (N=4). Significance was determined by a two-way ANOVA with Tukey post hoc test for multiple comparisons. *p<0.5, **p<0.01, ***p<1×10⁻⁴, ****p<1×10⁻⁵

FIGS. 3A-3F. Lipo^{TLR+I} generate antigen specific Treg in vivo and effective bystander tolerance. (FIGS. 3A-3C) C57BL/6 mice (4 per group) were injected with the following formulations: PBS control (PBS), OVA+free TLR agonists (TLR Free), OVA+Inhibitor free (I), liposomal OVA+TLR (Lipo^{TLR}) or liposomal OVA+Inhibitor combination (Lipo^{TLR+I}). Each formulation included combinations of 100 μg OVA/mouse, 10 μmol inhibitors/mouse, 1 μg FLA/mouse, 10 μg CpG/mouse. 10 days after last injection, serum was sampled and analyzed via ELISA for anti-OVA IgG. (FIG. 3A) On day 10, mice were sacrificed, their lymph nodes disassociated, the removed cells were stained and analyzed via flow. Lymphocytes were analyzed for the number of CD45+, CD3+, CD8−+, and MHCI OVA epitope tetramer positive cells were calculated. Representative flow plots of CD45+, CD3+, CD8+ cells showing distribution of CD8 (y axis) and major OVA MHCI tetramer signal (x axis). (FIG. 3B) Lymphocytes from the studies shown in FIG. 3A were also analyzed for the number of CD3+, CD4+, CD127−, FoxP3+, and MHCII OVA epitope tetramer positive cells (OVA specific T regs) were calculated. Below are representative flow plots of CD45+, CD3+, CD4+ cells showing distribution of major OVA MHCII tetramer signal (y axis) and FoxP3 (x axis). (FIG. 3D) Splenocytes from mice in the studies shown in FIG. 3A were isolated, stained with CFSE and allowed to incubate with BMDCs for 16 hrs (3:1 spleenocytes to BMDCs). The cell mixture was then incubated with either the major MHCI epitope (for CD8 cells) or MHCII epitope (for CD4 cells) from OVA for 48 hrs. T-cell proliferation of spleenocytes was assessed via CFSE assay for both CD4 and CD8 cells. (FIGS. 3E-3H) Liposome formulations similar to those used in the results shown in in FIG. 3A were loaded with either OVA (100 μg/mouse) or MOG₃₅₋₅₅ peptide (10 μg/mouse) and injected into C57Bl/6 mice on day 1 (FLA formulation) and day 2 (CpG formulation) (N=5). Mice were then either challenged with CpG/OVA (10 μg/mouse/100 μg/mouse) on day 5 or injected to induce MOG specific EAE disease on day 4 and 5 (see methods). See experiment schematic (lower right of FIG. 3E). On day 15, all mice were sacrificed, popliteal lymph nodes analyzed via flow cytometry for antigen specific T cell populations and blood analyzed for anti-MOG or OVA IgG titers. (FIG. 3E) OVA₃₂₃₋₃₃₉ MHCII tetramer+CD4+ T cell populations. (FIG. 3F) Anti-OVA IgG concentrations on day 15. (FIG. 3G) MOG₃₅₋₅₅ peptide MHCII tetramer+T reg cells. (FIG. 3H) MOG₃₅₋₅₅ peptide specific IgGs. Error bars indicate ± of SD of each mouse group (N=4-5). Significance was determined by a two-way ANOVA with Tukey post hoc test for multiple comparisons. *p<0.5, **p<0.01, ***p<1×10⁻⁴, ****p<1×10⁻⁵

FIGS. 4A-4F. Tolerogenic Liposomes Prevent EAE Disease Progression via Antigen Specific Bystander Tolerance. (FIG. 4A) C57BL/6 mice (15 mice per group) were injected on consecutive days to induce EAE. Following final injection, the disease was allowed to progress for 5 days, at which point mice were injected with treatment candidates on day 4 and 5, allowed to rest for 48 hrs then treated again on day 7 and 8. Treatment groups were: PBS (on both days), Free I, Lipo^{I}, or Lipo^{TLR+I}. For all TLR containing formulations, the FLA formulation was given first (e.g day 4), then 24 hrs later the CpG formulation was administered (e.g. day 5). All formulations (except PBS) contained 10 μg of MOG peptide in each 100 μL injection. MOG₃₅₋₅₅ peptide was fully encapsulated in liposomal formulations. After final treatment, mice were monitored for 37 days and disease progression tracked. Error bars represent ±SEM of disease score. (FIG. 4B) Groups of 5 mice were treated similarly to the studies shown in FIG. 4A, but sacrificed on day 14 following EAE induction. Serum was taken and analyzed for EAE peptide specific IgG levels via ELISA. UT denotes an “untreated” mouse, a naïve C57BL/6 mouse without MOG exposure. (FIG. 4C) Lymph nodes from mice used in the studies shown in FIG. 4B were dissociated and lymphocytes were stained for analysis of T-cells. The number of EAE peptide tetramer positive CD4+ activated T cells is shown. A representative flow plots of CD45+, CD3+, CD4+ cells shows a distribution of CD4 (y axis) and MOG₃₅₋₅₅ peptide tetramer (x axis). (FIG. 4D) EAE peptide tetramer positive T reg cells from lymph nodes from the studies shown in FIG. 4C. Shown are representative flow plots of CD45+, CD3+, CD4+ cells showing distribution of MHCII-MOG peptide tetramer (y axis) and FoxP3 (x axis). (FIGS. 4E-4G) C57Bl/6 mice (N=10) were similarly treated as in the studies shown in FIG. 4A with Lipo^{TLR+I}, but with treatment starting on day 8 after EAE induction. Mice were then injected i.p with PBS or antibodies against mouse (FIG. 4E) PD-L1, (FIG. 4F) PD-1 or (FIG. 4G) CTLA-4. Mice were injected with 500 μg antibody on day 7 and day 10 and 250 μg antibody on day 14 and day 17. Statistical significance of AUC differences of EAE disease development curves was assessed by using one-way ANOVA and Tukey's multiple comparison test in FIG. 4A and FIGS. 4E-4G. Significance for FIGS. 4B-4D was determined by a two-way ANOVA with Tukey post hoc test for multiple comparisons. *p<0.5, **p<0.01, ***p<1×10⁻⁴****p<1×10⁻⁵.

FIGS. 5A-5E. Combining two doses of inhibitors with FLA and CpG is the optimal formulation for inducing tolDCs. 1 million BMDCs per sample were incubated with 0.1 μg/mL FLA and/or inhibitors or just PBS on day 1. 16 hr later, cells were washed and incubated with 0.5 μM CpG or PBS with or without inhibitors. After 4 hrs supernatants were tested for TNFα and after 20 hrs tested for IL-10 (FIGS. 5D and 5E). Cell surface markers PD-L1 (FIG. 5A), CD80 (FIG. 5B) and CD40 (FIG. 5C) were tested after 20 hrs via flow cytometry. Error bars indicate of biological triplicate experiments.

FIGS. 6A-6C. Combination of tolerogenic drugs with TLR agonists shows increase in tolerance markers and decrease in inflammatory cell markers in mouse APC cell lines. 200 k of either RAW (left) or DC2.4 (right) cells were treated similarly as in the studies shown in FIG. 1B, then analyzed via flow for (FIG. 6A) PD-L1, (FIG. 6B) CD80, (FIG. 6C) CD40. N=3 and error bars indicate SD.

FIGS. 7A-7C. Altering ratios of inhibitors induces varying levels of tolDC phenotypes. 200 k BMDCs were treated as in FIG. 1B (FLA followed by CpG treatment) varying by the ratios of Dex, Sim and SC-514 at either a 10 μM (left) or 1 μM (right) total inhibitor concentration. BMDCs were analyzed by flow cytometry for (FIG. 7A) CD40, (FIG. 7B) CD80, (FIG. 7C) PD-L1. N=3 and error bars indicate SD.

FIGS. 8A-8E. Synthesis of FLA and CpG lipids and characterization of liposomes. (FIG. 8A) Synthesis schematic of CpG-lipid conjugate (top) and FLA-lipid conjugate (bottom). (FIG. 8B) Gel electrophoresis of CpG (left) and FLA (right) lipid conjugates. GpG lipid was run on a 2.5% agarose-TBE gel and stained with Sybr Safe DNA stain (thermo). FLA lipid was run on a 4-15% Mini-PROTEAN TGX precast gel (Bio-Rad) and stained with coomassie blue. Red arrows indicate TLR agonist lipid conjugates with slight increases in MW. (FIG. 8C) Table showing dynamic light scattering (DLS) data for 200 nm, 50 mM total lipid liposomes made with 0.33 mM of each inhibitor, 1 mg/mL OVA and either 10 μg/mL FLA-lipid or 100 μg/mL CpG or none (Blank) as described from the in vivo study in FIG. 2. (FIG. 8D) Liposomes from the studies shown in FIG. 8C were tested via HPLC against standard injections and a percent liposomal loading calculated for each inhibitor and TLR agonist. (FIG. 8E) OVA containing liposomes from FIG. 8C, a blank OVA liposome and MOG peptide containing liposomes were similarly analyzed via HPLC and compared to a standard injection of OVA or MOG and a percent loading calculated.

FIGS. 9A-9B. Inhibitors packaged in liposomes have induce a similar tolDC phenotype in vitro as free inhibitors. 1 million BMDCs were incubated with FLA+I then 16 hrs later treated with CpG+I, then 20 hrs later analyzed via flow. Cells were treated with 0.2 μg/mL FLA or 1 μM CpG with 20 μM of total inhibitors (1:1:1 ratio) and serially diluted (concentrations indicate total inhibitor concentrations). (FIG. 9A) free I (FIG. 9B) LipoTLR+I. N=3 and error bars indicate SD.

FIGS. 10A-10D. OVA bearing tolerogenic liposomes have high levels of antigen presentation. 1 million BMDCs were treated with AF647 labeled OVA at 10 μg/mL (OVA), or OVA+agonist at 0.1 μg/mL FLA and 0.5 μM CpG (Free TLR), inhibitors at 10 μM total inhibitor concentration+OVA (Free I), OVA+agonist+inhibitor (Free TLR+I) or liposomal versions of each of these. Cells were treated with these formulations or 10× and 100× dilutions of each. Cells were treated with FLA formulations then CpG/OVA formulations (note that OVA was only added in CpG treatment). 1 hr after CpG/OVA treatment, cells were analyzed via flow cytometry for (FIG. 10A) major OVA MHCI-I peptide-MHCI expression total signal, (FIG. 10B) percent MHCI-peptide positive cells, (FIG. 10C) OVA signal and (FIG. 10D) OVA positive cells. Error bars indicate ± of biological triplicate experiments.

FIG. 11. Gating Strategy of Tolerogenic Liposome treated mouse lymphocytes. Lymphocytes from treated mice from studies shown in FIGS. 2B-2G were gated for live cells on FSC and SSC, then gated for single cells on SSC-A and SSC-H (not shown), gated on lymphocytes by DAPI and CD45 intensity, then gated for DCs on MHCII+ and CD11c+. These cells were gated on CD103, CD40, CD80, CD86, PD-L1, and PD-L2.

FIGS. 12A-12C. T cell analysis of mouse lymph nodes from experiment in FIG. 3B,C. (FIG. 12A) Gating strategy for lymphocytes identifying MHCII-epitope tetramer+ Tregs. (FIG. 12B) Cell counts of live lymphocytes from various treatment groups. (FIG. 12C) T cell distribution of lymphocytes for various treatment groups. Error bars indicate ±SD of N=3-4 mice per group.

FIG. 13. Cultured Splenocytes taken from mice treated with tolerogenic liposomes reduce INFγ production. Splenocytes described in FIG. 3E were tested for INFγ production using via CBA in a similar fashion as IL-2. Error bars indicate ±SD of N=3-4 mice per group.

FIGS. 14A-14H. Lipo^{TLR+I} suppresses antigen specific T cell responses with minimal disruption of other antigen specific responses. Mice treated as shown in FIGS. 3E-3H with MOG or OVA specific liposomes and then challenged with either EAE or OVA/CpG were further analyzed. Lymph cells from these mice were analyzed for (FIG. 14A) populations of CD4, CD8 T cells and B cells and show no change (FIG. 14B) total Treg populations, showing a slight but insignificant increase in Lipo^{TLR+I} treated groups. (FIG. 14C) Lymph cells were analyzed with tetramers for the major MHCI and MHCII OVA epitopes, showing no decrease for MOG-Lipo^{TLR+I} treatment groups for either epitope. (FIG. 14D) Similarly there was no decrease in MOG tetramer positive cells between NT and OVA-Lipo^{TLR+I} groups indicating no reduction in antigen specific T cells for perpendicular treatment groups. (FIGS. 14E-14H). Splenocytes from these mice were analyzed with ICS for antigen specific T cell responses. 1 million splenocytes were incubated with either 10 μg/mL of OVA₂₅₇₋₂₆₈ (OVAI), OVA₃₂₃₋₃₃₉ (OVAII) or MOG₃₅₋₅₅ (EAE) for 6 hours with brefeldin A and monensin (BD biosciences). Cells were then fixed and analyzed for (FIG. 14E) CD4+, INFγ+(FIG. 14F) CD4+, IL4+, (FIG. 14G) CD4+, IL17A+, or (FIG. 14H) CD8+, INFγ+ T cells. Error bars indicate ±SD of N=3-4 mice per group.

FIGS. 15A-15B. EAE testing optimization. Mice (N=10 per group) were treated 3 days prior with PBS or an injection of EAE peptide (10 μg per mouse) and free combination of inhibitors for a total of 10 μmols. EAE was induced with either (FIG. 15A) 200 ng or (FIG. 15B) 100 ng to determine optimal formulation. 200 ng was used in EAE studies described in the Examples herein.

FIG. 16. Selecting new PPI formulations from large screening data. Primary screen data of IRF3 vs NF-kB fold increase (compared to controls with no inhibitor) for >50,000 unique combinations showing selection regions in red. Paired down selection regions with middle IRF/low NF-kB and low IRF/NF-kB to get top 500 combinations. Combinations were further paired down in a secondary screen using THP-1 cells and observing released cytokines of TNFα, IL-6, IP-10, IL-1β, IL-12 via AlphaELISA and a cytokine score obtained by calculating fold cytokine increase relative to TLR agonists alone and then adding all 5 cytokines and multiplying by 1000. Viability was assessed via FSC and SSC using flow.

FIG. 17. Top 10 new PPI candidates. Preliminary screening of top 389 combinations. 500 k BMDCs were incubated with combinations overnight then tested for IL-10 and TNFα secretion and plotted. N=3. Top 10 combinations with highest IL-10/TNFα are plotted in red.

FIG. 18. Top 10 new PPI candidates. Preliminary screening of top 389 combinations. 500 k BMDCs were incubated with combinations overnight then tested for IL-10 and TNFα secretion and viability using MTT assay and plotted. N=3. Top 10 combinations with highest IL-10/TNFα are plotted in red that were viable. These top 10 combinations are shown in the table below:

TABLE 3
List of top performing inhibitor compounds and associated
agonists. Note all compounds were tested at 1 uM.

| Compound | TLR Agonist |
|---|---|
| Cucurbitacin I | LPS (10 nM) |
| Costunolide | Pam2 (100 nM) |
| MLN120B | Pam2 (100 nM) |
| (−)-Parthenolide | Pam2 (100 nM) |
| Peficitinib | Pam2 (100 nM) |
| Oclacitinib maleate | R848 (2.5 ug/mL) |
| AD80 | Pam2 (100 nM) |
| Cucurbitacin B | R848 (2.5 ug/mL) |
| CEP-33779 | R848 (2.5 ug/mL) |
| Dehydrocostus Lactone | R848 (2.5 ug/mL) |

### DETAILED DESCRIPTION OF THE INVENTION

The present disclosure is based, at least in part, on the development of a new push/pull method for inducing antigen-specific Treg responses via induction of tolerogenic dendritic cells (tolDCs), as well as compositions useful in such methods. As disclosed herein, compositions comprising an antigen and an immunomodulator, together with an immune activator such as a toll-like receptor (TLR) agonist, serve to generate tolDCs and stimulate production of antigen-specific Tregs. As further described herein, a cocktail of three immunomodulators—dexamethasone, simvastatin, and SC-514—works synergistically to generate tolDCs when used in combination with one or more TLR agonists. When the tolerogenic combination is packaged into a liposome with an antigen, these tolDCs induce differentiation of antigen-specific Tregs both ex vivo and in vivo.

### I. Induction of Tolerogenic Dendritic Cells

As used herein, “Dendritic Cells” (DCs) refers to any member of a diverse population of morphologically similar cell types found in lymphoid or non-lymphoid tissues. Without wishing to be bound by theory, these cells are understood to be characterized by their distinctive morphology and surface MHC-class II expression (Steinman, et al., Ann. Rev. Immunol. 9:271 (1991); Schraml, et al., Current opinion in immunology, 32, 13-20 (2015); both incorporated herein by reference for their description of such cells). Tolerogenic DCs (tolDCs) describe a specialized DCs subset which overexpress tolerance markers such as PD-L1/2, lack T cell co-stimulatory markers such as CD80, CD86 or CD40, and release tolerogenic cytokines such as IL-10.

The present disclosure describes methods and systems for generating tolerogenic antigen presenting cells, such as tolerogenic dendritic cells (tolDCs), to facilitate development of antigen-specific T regulatory cells (Tregs; also “regulatory T cells”). The disclosure describes in vivo generation of tolDCs by administration of an antigen, a TLR agonist (e.g., a TLR5 or TLR9 agonist), and one or more immunosuppressive agents, thereby facilitating development of Tregs specific for the antigen. As used herein, an “immunosuppressive agent” (also “immunomodulator”) describes any molecule capable, alone or in combination with other agents, of suppressing an immune response in a subject. In one example, an immunosuppressive agent is be a molecule which, when administered to cells in combination with a TLR agonist, reduces the severity of an immune response to the TLR agonist compared with administration of the TLR agonist alone. In another example, as disclosed herein, an immunosuppressive agent is a molecule (e.g., small molecule, nucleic acid, polypeptide, etc.) which, when administered to a subject with a TLR agonist (e.g., a TLR5 agonist or TLR9 agonist) and an antigen, serves to reduce the severity and/or frequency of an immune response to the antigen in the subject. Methods and compositions of the present disclosure may include at least, at most, or exactly 1, 2, 3, 4, 5, 6, 7, 8, 9, or 10 immunosuppressive agents. Non-limiting examples of immunosuppressive agents contemplated herein include dexamethasone, rapamycin, Ridaforolimus, acetylsalicylic acid, ly294002, simvastatin, IRF3-siRNA, 3′-Sialyl Lewis X, Cholecalciferol (Vitamin D3), Vasoactive intestinal peptide, Lifitegrast, TSLP, minocycline, Hydroxychloroquine, Ligustilide, Sapanisertib, HGF mouse protein, Retinoic acid, BX-795, SB203580, SP600125, Curcumin, Bee venom PLA2, PI103, Parthenolide, and SC 514 (also known as GK 01140, 4-Amino-[2,3′-bithiophene]-5-carboxamidee, or 3-Amino-5-(3-thiophenyl)-2-thiophenecarboxamide). An immunosuppressive agent of the disclosure comprises dexamethasone. An immunosuppressive agent of the disclosure comprises simvastatin. An immunosuppressive agent of the disclosure may comprise SC 514.

The disclosure may comprise tolerogenic compositions comprising a TLR agonist and one or more immunosuppressive agents, as well as use of such compositions in therapeutic methods, such as methods for treatment of an autoimmune condition. In some cases a tolerogenic composition additionally comprises an antigen. Accordingly, disclosed are compositions, including pharmaceutical compositions, comprising an antigen, a TLR agonist, and one or more immunosuppressive agents. A tolerogenic composition of the disclosure may comprise 1, 2, 3, 4, 5, 6, 7, 8, 9, or 10 immunosuppressive agents, or more. A composition of the disclosure may comprise one or more of dexamethasone, rapamycin, Ridaforolimus, acetylsalicylic acid, ly294002, simvastatin, IRF3-siRNA, 3-Sialyl Lewis X, Cholecalciferol (Vitamin D3), Vasoactive intestinal peptide, Lifitegrast, TSLP, minocycline, Hydroxychloroquine, Ligustilide, Sapanisertib, HGF mouse protein, Retinoic acid, BX-795, SB203580, SP600125, Curcumin, Bee venom PLA2, PI103, Parthenolide, and SC 514. A composition of the disclosure may comprise three immunosuppressive agents. A composition of the disclosure may comprise dexamethasone, simvastatin, and SC 514. A composition of the disclosure may comprise dexamethasone, simvastatin, and SC 514 at a ratio of 1:1:1. A composition of the disclosure may comprise dexamethasone, simvastatin, and SC 514 at different concentrations. Also disclosed are methods comprising providing such compositions to immune cells. Compositions of the disclosure may be provided to immune cells in vitro, ex vivo, and/or in vivo. A composition of the disclosure may be administered to a population of cells (e.g., dendritic cells or dendritic cell precursors) in vitro. A composition of the disclosure may be administered to a subject in vivo. The immune cells may be antigen presenting cells. Examples of antigen presenting cells contemplated herein include dendritic cells, macrophages, and B-cells. Antigen presenting cells of the disclosure may be dendritic cells (DCs). Administration of such a tolerogenic composition to antigen presenting cells may serve to generate tolerogenic antigen presenting cells, thereby reducing an immune response at least in part by facilitating formation of T regulatory cells (Tregs).

An immunogenic composition of the disclosure may comprise a nanocarrier comprising 1) an antigen; 2) a TLR agonist, and 3) one or more immunosuppressive agents. As used herein, a “nanocarrier” describes a particle configured to carry and deliver one or more molecules, where the particle has a diameter of less than 1000 nm. A nanocarrier of the present disclosure may have a diameter of at most, at least, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, or 500 nm, or any range or value therein. A nanocarrier of the disclosure may have a diameter of less than 300 nm. A nanocarrier of the disclosure may have a diameter of less than 200 nm. Various types of nanocarriers are contemplated herein, including, for example, liposomes, polymeric nanoparticles, polymeric micelles, silica nanoparticles, and solid lipid nanoparticles. A nanocarrier of the present disclosure may be further defined as a liposome. A nanocarrier of the disclosure may comprise dexamethasone, simvastatin, and SC 514 at a ratio of 1:1:1. A nanocarrier of the disclosure may comprise dexamethasone, simvastatin, and SC 514 at different concentrations.

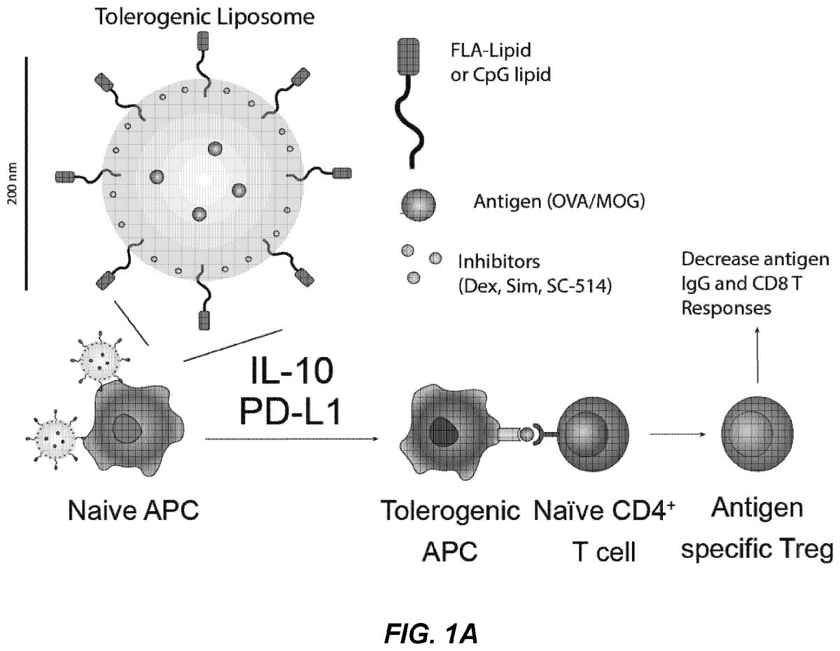

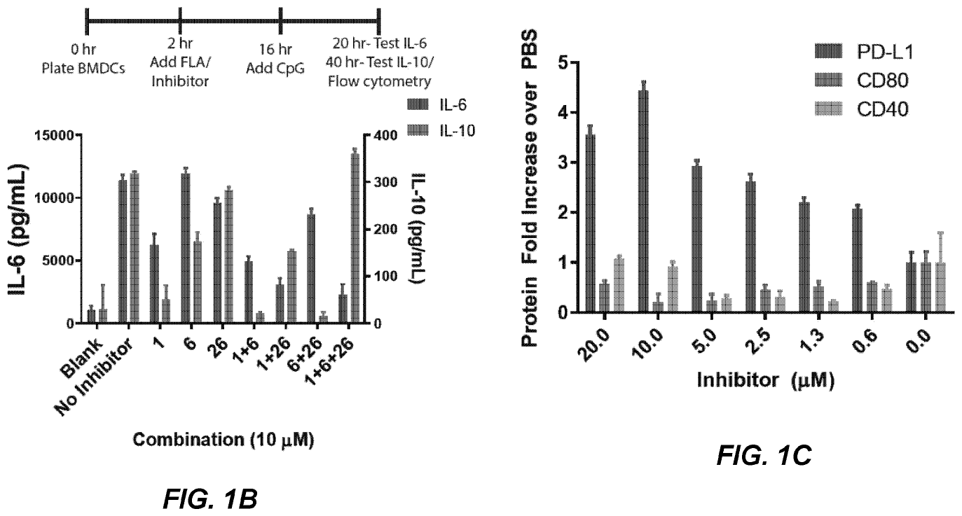

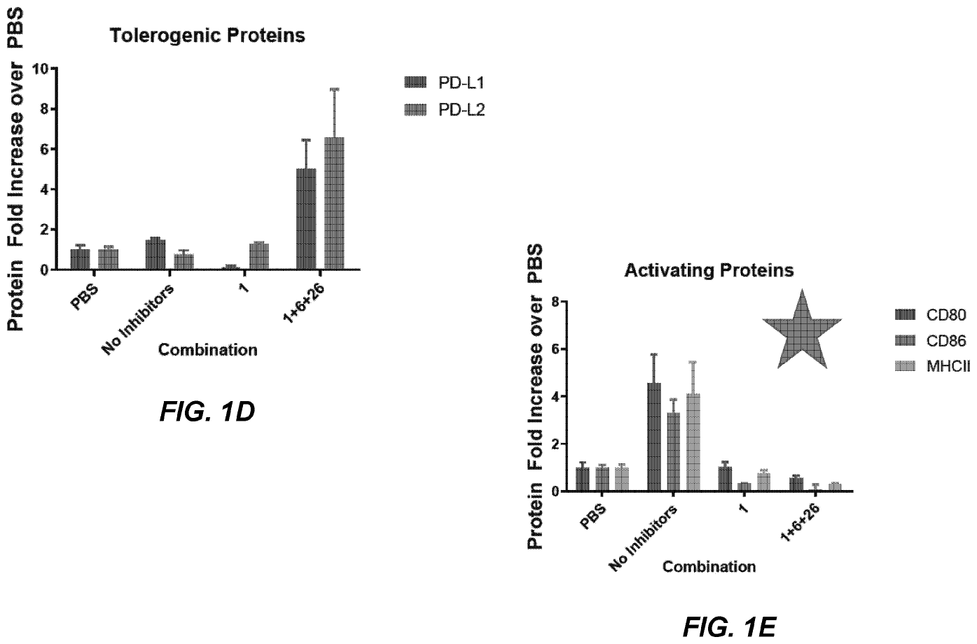

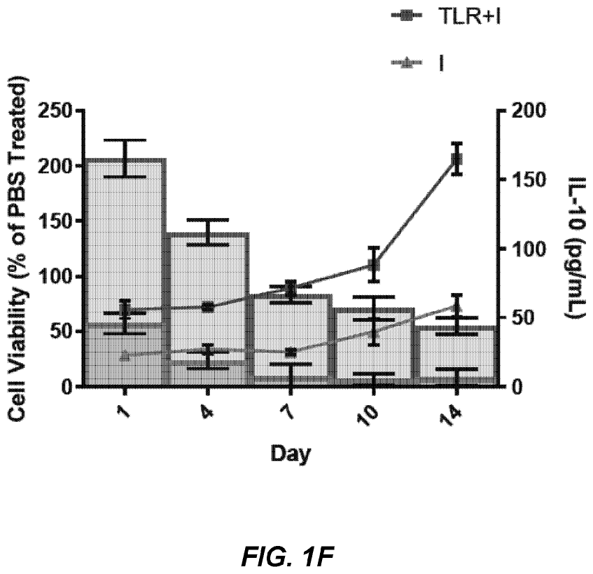

A liposome of the present disclosure may comprise a molecule in an interior of the liposome (e.g., wherein the molecule is a hydrophilic molecule such as an antigenic peptide), attached to the surface of the liposome (e.g., via attachment of the molecule to a lipid), and/or within a membrane (i.e., lipid bilayer) of the liposome (e.g., where the molecule is a hydrophobic molecule). A liposome of the disclosure may comprise 1) an antigen; 2) a TLR agonist, and 3) one or more immunosuppressive agents. Accordingly, a liposome may comprise an antigen (e.g., a peptide antigen) encapsulated in an interior of the liposome, a TLR agonist (e.g., TLR5 agonist or TLR9 agonist) conjugated to a lipid and presented on the surface of the liposome, and one or more immunosuppressive agents (e.g., dexamethasone, simvastatin, and/or SC 514) encapsulated within a membrane of the liposome (see, e.g., FIG. 1A for a single, non-limiting example of a liposome composition of the disclosure). A liposome of the disclosure may comprise 1) an antigen associated with an autoimmune condition encapsulated in an interior of the liposome, 2) a TLR5 agonist (e.g., flagellin) conjugated to a lipid and presented on the surface of the liposome, and 3) dexamethasone, simvastatin, and SC 514 encapsulated within a membrane of the liposome. A liposome of the disclosure may comprise 1) an antigen associated with an autoimmune condition encapsulated in an interior of the liposome, 2) a TLR9 agonist (e.g., a CpG oligonucleotide) conjugated to a lipid and presented on the surface of the liposome, and 3) dexamethasone, simvastatin, and SC 514 encapsulated within a membrane of the liposome.

Described herein are methods for treatment of an autoimmune condition in a subject comprising administering one or more tolerogenic compositions (e.g., nanocarriers comprising an antigen, TLR agonist, and immunosuppressive agent) or combinations of TLR agonist and immunosuppressive agent to a subject having or at risk for an autoimmune condition. A method of the disclosure may comprise administration of 1, 2, 3, 4, 5, 6, or more tolerogenic compositions.

II. PRR agonists and TLR agonists

The present disclosure relates to compositions, methods, and nanocarriers comprising pattern recognition receptor (PRR) agonists. A PRR agonist may be any molecule that, directly or indirectly, activates a PRR or stimulates PRR signaling. PRRs include cell surface receptors (e.g., toll-like receptor (TLR) agonists) and intracellular receptors (e.g., RIG-I-like receptors). Examples of PRRs targeted by agonists of the present disclosure include NOD-like receptors, RIG-I-like receptors, STING receptors, and toll-like receptors (TLRs). Disclosed herein are PRR agonists, wherein a PRR agonist is a NOD-like receptor agonist, a RIG-I-like receptor agonist, a STING agonist, or a TLR agonist. A PRR agonist may be further defined as a TLR agonist.

The present disclosure relates to compositions, methods, and nanocarriers comprising TLR agonists. A TLR agonist may be any molecule that, directly or indirectly, activates a TLR and/or stimulates TLR signaling. In some cases, a TLR agonist is a molecule that binds directly to a TLR. TLR agonists may be formulated into nanocarriers, such as liposomes, in some cases together with an antigen (e.g., peptide antigen) and/or one or more immunosuppressive agents. In some cases, a TLR agonist of the present disclosure is conjugated, for example, to a lipid for incorporation into a liposome or other nanocarrier.

The TLR agonist may be one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111:B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam2CSK4; lipoarabinomannan or lipomannan from M. smegmatis; triacylated lipoproteins such as Pam3CSK4; lipoproteins such as MALP-2 and MALP-404 from mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™ Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CL097, 2Bxy, R848, or R848 VacciGrade™) TLR9 (e.g., CpG ODNs such as CpG ODN 1826); and TLR11 (e.g., Toxoplasma gondii Profilin). The TLR agonist may be an amphiphilic TLR agonist. The TLR agonist may be a TLR 2/6 agonist, for example Pam2CSK4 or Pam3CSK4. The TLR agonist may be a hydrophobic TLR agonist. The TLR agonist may be a TLR 7, TLR 8, or TLR 7/8 agonist, for example 2Bxy. The TLR agonist may be a specific agonist listed above. The TLR agonist may be one that agonizes either one TLR or two TLRs specifically.

Disclosed herein are small molecule compounds suitable for use as TLR agonists. Examples of small molecule TLR agonists include compounds having a 2-aminopyridine fused to a five membered nitrogen-containing heterocyclic ring. Such compounds include, for example, imidazoquinoline amines including but not limited to substituted imidazoquinoline amines such as, for example, aminoalkyl-substituted imidazoquinoline amines, amide-substituted imidazoquinoline amines, sulfonamide-substituted imidazoquinoline amines, urea-substituted imidazoquinoline amines, aryl ether-substituted imidazoquinoline amines, heterocyclic ether-substituted imidazoquinoline amines, amido ether-substituted imidazoquinoline amines, sulfonamido ether-substituted imidazoquinoline amines, urea-substituted imidazoquinoline ethers, and thioether-substituted imidazoquinoline amines; tetrahydroimidazoquinoline amines including but not limited to amide-substituted tetrahydroimidazoquinoline amines, sulfonamide-substituted tetrahydroimidazoquinoline amines, urea-substituted tetrahydroimidazoquinoline amines, aryl ether-substituted tetrahydroimidazoquinoline amines, heterocyclic ether-substituted tetrahydroimidazoquinoline amines, amido ether-substituted tetrahydroimidazoquinoline amines, sulfonamido ether-substituted tetrahydroimidazoquinoline amines, urea-substituted tetrahydroimidazoquinoline ethers, and thioether-substituted tetrahydroimidazoquinoline amines; imidazopyridine amines including but not limited to amide-substituted imidazopyridine amines, sulfonamido-substituted imidazopyridine amines, urea-substituted imidazopyridine amines; aryl ether-substituted imidazopyridine amines, heterocyclic ether-substituted imidazopyridine amines, amido ether-substituted imidazopyridine amines, sulfonamido ether-substituted imidazopyridine amines, urea-substituted imidazopyridine ethers, and thioether-substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines.

The TLR agonist may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

The TLR agonist may be a sulfonamide-substituted imidazoquinoline amine. The TLR agonist can be a urea-substituted imidazoquinoline ether. In another alternative embodiment, the TLR agonist can be an aminoalkyl-substituted imidazoquinoline amine. In one particular embodiment, the TLR agonist is 4-amino-α,α,2-trimethyl-1H-imidazo[4,5-c]quinolin-1-ethanol. In an alternative particular embodiment, the TLR agonist is N-(2-{2-[4-amino-2-(2-methoxyethyl)-1H-imidazo[4,5-c]quinolin-1-yl] ethoxy} ethyl)-N-methylmorpholine-4-carboxamide. In another alternative embodiment, the TLR agonist is 1-(2-amino-2-methylpropyl)-2-(ethoxymethyl)-1H-imidazo[4,5-c]quinolin-4-amine. In another alternative embodiment, the TLR agonist is N-[4-(4-an-no-2-ethyl-1H-imidazo[4,5-c]quinolin-1-yl)butyl]methanesulfonamide. In yet another alternative embodiment, the TLR agonist is N-[4-(4-amino-2-propyl-1H-imidazo[4,5-c]quinolin-1-yl)butyl]methanesulfonamide.

The TLR agonist may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an aminoalkyl-substituted imidazoquinoline amine, an amide-substituted imidazoquinoline amine, a sulfonamide-substituted imidazoquinoline amine, a urea-substituted imidazoquinoline amine, an aryl ether-substituted imidazoquinoline amine, a heterocyclic ether-substituted imidazoquinoline amine, an amido ether-substituted imidazoquinoline amine, a sulfonamido ether-substituted imidazoquinoline amine, a urea-substituted imidazoquinoline ether, or a thioether-substituted imidazoquinoline amines.

### III. Autoimmune or Inflammatory Conditions

Methods of the disclosure relate to the treatment of autoimmune or inflammatory conditions, and methods and compositions for treatment and/or prevention thereof. An autoimmune or inflammatory condition describes conditions characterized by an overactive immune response, either against a self-antigen (e.g., a myelin sheath protein, leading to multiple sclerosis) or a foreign antigen (e.g., a transplant graft, leading to graft versus host disease or graft rejection). The disclosed methods may comprise treatment of an autoimmune condition using a composition comprising an antigen (e.g., an autoimmune antigen), a TLR agonist, and one or more immunosuppressive agents (e.g., one or more immunomodulators of Table 1, such as dexamethasone, simvastatin, SC 514, ly294002, minocycline, hydroxychloroquine, Sialyl Lewis X, Thymic Stromal Lymphopoietin (TSLP), and/or lifitegrast). Also disclosed are compositions comprising an antigen associated with an autoimmune or inflammatory condition.

The autoimmune condition or inflammatory condition amenable for treatment or prevention may include, but not be limited to conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

An autoimmune inflammatory condition of the present disclosure may include multiple sclerosis. An autoimmune or inflammatory condition of the present disclosure may include graft versus host disease. An autoimmune or inflammatory condition of the present disclosure may be graft rejection. An autoimmune or inflammatory condition of the present disclosure may be arthritis (e.g., rheumatoid arthritis). An autoimmune or inflammatory condition of the present disclosure may be lupus.

### IV. Cancer Therapy

The disclosed methods may comprise administering a cancer therapy to a subject or patient. The cancer therapy may comprise a local cancer therapy. The cancer therapy may exclude a systemic cancer therapy. The cancer therapy may exclude a local therapy. The cancer therapy may comprise a local cancer therapy without the administration of a system cancer therapy. The cancer therapy may comprise administering a dimer of the present disclosure. The cancer therapy may comprise a radiotherapy. The cancer therapy may comprise a chemotherapy. The cancer therapy may comprise an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. The cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. The cancer may be a Stage I cancer. The cancer may be a Stage II cancer. The cancer may be a Stage III cancer. The cancer may be a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Disclosed are methods for treating cancer originating from the colon. The cancer may be colon cancer. The cancer may be colorectal cancer.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### Radiotherapy

Radiotherapy, such as ionizing radiation, may be administered to a subject. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

The radiotherapy can comprise external radiotherapy, internal radiotherapy, radioimmunotherapy, or intraoperative radiation therapy (IORT). The external radiotherapy may comprise three-dimensional conformal radiation therapy (3D-CRT), intensity modulated radiation therapy (IMRT), proton beam therapy, image-guided radiation therapy (IGRT), or stereotactic radiation therapy. The internal radiotherapy may comprise interstitial brachytherapy, intracavitary brachytherapy, or intraluminal radiation therapy. The radiotherapy may be administered to a primary tumor.

The amount of ionizing radiation is greater than 20 Gy and may be administered in one dose. The amount of ionizing radiation may be 18 Gy and is administered in three doses. The amount of ionizing radiation may be at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). The ionizing radiation may be administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of radiotherapy administered to a subject may be presented as a total dose of radiotherapy, which is then administered in fractionated doses. For example, the total dose may be 50 Gy administered in 10 fractionated doses of 5 Gy each. The total dose may be 50-90 Gy, administered in 20-60 fractionated doses of 2-3 Gy each. The total dose of radiation may be at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses may be administered (or any derivable range therein). At least, at most, or exactly 1, 2, 3, 4, 5, 6, 7, 8, 9, 10, 11, or 12 (or any derivable range therein) fractionated doses may be administered per day. At least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses may be administered per week.

### A. Cancer Immunotherapy

The methods may comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

The methods and compositions of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-i” include CD279 and SLEB2. Alternative names for “PDL1” include B7-H1, B7-4, CD274, and B7-H. Alternative names for “PDL2” include B7-DC, Btdc, and CD273. PD-1, PDL1, and PDL2 may be human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may be PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MED10680, also known as AMP-514, and REGN2810.

The immune checkpoint inhibitor may be a PDL1 inhibitor such as Durvalumab, also known as MED14736, atezolizumab, also known as MPDL3280A, avelumab, also known as MSB00010118C, MDX-1105, BMS-936559, or combinations thereof. The immune checkpoint inhibitor may be a PDL2 inhibitor such as rHIgM12B7.

The inhibitor may comprise the heavy and light chain CDRs or VRs of nivolumab, pembrolizumab, or pidilizumab. The inhibitor may comprise the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may be one that blocks the CTLA-4 and B7-1 interaction. The inhibitor may be one that blocks the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

The inhibitor may comprise the heavy and light chain CDRs or VRs of tremelimumab or ipilimumab. The inhibitor may comprise the CDR1, CDR2, and CDR3 domains of the VH region of tremelimumab or ipilimumab, and the CDR1, CDR2 and CDR3 domains of the VL region of tremelimumab or ipilimumab. The antibody may be one that competes for binding with and/or binds to the same epitope on PD-1, B7-1, or B7-2 as the above-mentioned antibodies. The antibody may have at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

The immune checkpoint inhibitor may be an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (EVIP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

The inhibitor may comprise the heavy and light chain CDRs or VRs of an anti-LAG3 antibody. The inhibitor may comprise the CDR1, CDR2, and CDR3 domains of the VH region of an anti-LAG3 antibody, and the CDR1, CDR2 and CDR3 domains of the VL region of an anti-LAG3 antibody. The antibody may have at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

The immune checkpoint inhibitor may be an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

The inhibitor may comprise the heavy and light chain CDRs or VRs of an anti-TIM-3 antibody. The inhibitor may comprise the CDR1, CDR2, and CDR3 domains of the VH region of an anti-TIM-3 antibody, and the CDR1, CDR2 and CDR3 domains of the VL region of an anti-TIM-3 antibody. The antibody may have at least about 70, 75, 80, 85, 90, 95, 97, or 99% (or any derivable range or value therein) variable region amino acid sequence identity with the above-mentioned antibodies.

### 2. Activation of Co-Stimulatory Molecules

The immunotherapy may comprise an activator of a co-stimulatory molecule. The activator may comprise an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TTLs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Methods and compositions of the disclosure include patients that have been previously treated for a therapy described herein, are currently being treated for a therapy described herein, or have not been treated for a therapy described herein. The patient may be one that has been determined to be resistant to a therapy described herein. The patient may be one that has been determined to be sensitive to a therapy described herein. For example, the patient may be one that has been determined to be sensitive to an immune checkpoint inhibitor therapy based on a determination that the patient has or previously had pancreatitis.

### B. Chemotherapies

The additional therapy may comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be used as a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### C. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### V. Antigens

The term “antigen” as used herein refers to a molecule against which a subject can initiate a humoral and/or cellular immune response. Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. The antigen may be an antigen involved in an autoimmune disease. In certain compositions and methods of the disclosure, the antigen is a peptide.

Further examples of antigens useful in the methods and compositions of the disclosure are provided below and throughout the disclosure.

### A. Antigens Relating to Autoimmunity

Antigens involved in autoimmune diseases, allergy, and graft rejection can be used in the compositions and methods of the disclosure. For example, an antigen involved in (or “associated with”) any one or more of the following autoimmune diseases or disorders can be used in the present disclosure: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, bone marrow, and neural graft components.

In addition to full length proteins, antigens also describe immunogenic portions of proteins, such as peptides comprising a portion of a sequence from a protein. For example, an antigen of the disclosure for use in treating an autoimmune condition may be a peptide having a sequence of a portion of a protein associated with the autoimmune condition. The autoimmune condition may be multiple sclerosis. Accordingly, in such cases, an antigen of the compositions and methods of the present disclosure may be a peptide from a protein associated with multiple sclerosis, such as a myelin sheath protein or portion thereof. Example myelin sheath proteins from include myelin basic protein (MBP), myelin oligodendrocyte glycoprotein (MOG), proteolipid protein (PLP), myelin associated glycoprotein (MAG), peripheral myelin protein (PMP-22), P₀ protein, connexin 32 protein, Schwann cell myelin protein, and oligodendrocyte-myelin glycoprotein (OMgp). An antigen of the disclosure may comprise an MBP peptide, an MOG peptide, a PLP peptide, or an MAG peptide. An antigen of the disclosure may comprise a MOG peptide. It is contemplated that one or more of the antigens and antigenic components listed in this section are specifically excluded in certain aspects of the disclosure.

### VI. Adjuvants

Aspects of the present disclosure include adjuvants and methods for administering adjuvants to a subject. The immunogenicity of a particular composition can be enhanced by the use of non-specific stimulators of the immune response, known as adjuvants. “Adjuvant” as used herein refers to a substance, which when administered prior, together or after administration of an antigen, accelerates, prolongs and/or enhances the quality and/or strength of an immune response to the antigen in comparison to the administration of the antigen alone. Adjuvants that may be used in accordance with aspects include, but are not limited to, IL-1, IL-2, IL-4, IL-7, IL-12, y-interferon, GM-CSF, BCG, aluminum hydroxide, MDP compounds, such as thur-MDP and nor-MDP, CGP (MTP-PE), lipid A, and monophosphoryl lipid A (MPL). Other example adjuvants may include complete Freund's adjuvant (a non-specific stimulator of the immune response containing killed Mycobacterium tuberculosis), incomplete Freund's adjuvants, and/or aluminum hydroxide adjuvant.

In some aspects, an adjuvant of the disclosure is a TLR agonist. Use of TLR agonists as adjuvants is described in, for example, Li et al., TLR Agonists as Adjuvants for Cancer Vaccines. Adv Exp Med Biol. 2017; 1024:195-212 (incorporated herein by reference in its entirety).

### VII. Administration of Therapeutic Compositions

The therapy provided herein may comprise administration of a combination of therapeutic agents, such as a first immunogenic composition (e.g., first nanocarrier comprising an antigen; a TLR agonist; and one or more immunosuppressive agents) and a second immunogenic composition (e.g., second nanocarrier comprising an antigen; a TLR agonist; and one or more immunosuppressive agents). The therapies may be administered in any suitable manner known in the art. For example, the first and second immunogenic compositions may be administered sequentially (at different times) or concurrently (at the same time). In some aspects, the first and second cancer immunogenic compositions are administered in a separate composition. In some aspects, the first and second immunogenic compositions are in the same composition. In some aspects, a first immunogenic composition is a nanocarrier comprising an antigen, a TLR5 agonist, (e.g., flagellin) and one or more immunosuppressive agents (e.g., dexamethasone, simvastatin, and SC 514). In some aspects, a second immunogenic composition is a nanocarrier comprising an antigen; a TLR9 agonist (e.g., a CpG oligonucleotide); and one or more immunosuppressive agents (e.g., dexamethasone, simvastatin, and SC 514).

In some aspects, the first immunogenic composition and the second immunogenic composition are administered substantially simultaneously. In some aspects, the first immunogenic composition and the second immunogenic composition are administered sequentially. In some aspects, the first immunogenic composition, the second immunogenic composition, and a third immunogenic composition are administered sequentially. In some aspects, the first immunogenic composition is administered before administering the second immunogenic composition. In some aspects, the first immunogenic composition is administered after administering the second immunogenic composition. In some aspects, the second immunogenic composition is administered after administering the first immunogenic composition. In some aspects, a first immunogenic composition is administered to a subject comprising a nanocarrier comprising 1) an antigen, 2) a TLR5 agonist, and 3) dexamethasone, simvastatin, and SC 514; following this, a second immunogenic composition is administered to a subject comprising a nanocarrier comprising 1) an antigen, 2) a TLR9 agonist, and 3) dexamethasone, simvastatin, and SC 514. In certain aspects, the second immunogenic composition is administered at least 6, 7, 8, 9, 10, 11, 12, 13, 14, 15, 16, 17, 18, 19, 20, 21, 22, 23, 24, 28, 32, 36, 40, 44, 48, 72, or 96 hours (or any range or value derivable therein) after administering the first immunogenic composition. In some aspects, the second immunogenic composition is administered at least 1, 2, 3, 4, or 5 days following administration of the first immunogenic composition. In some aspects, the second immunogenic composition is administered the day after administration of the first immunogenic composition.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, example methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### VIII. Cellular Therapies

Certain aspects of the disclosure relate to therapies involving delivery of cells to a subject. Such cellular therapies may include, for example, administration of tolerogenic dendritic cells generated ex vivo using certain compositions or methods of the disclosure.

### A. Cell Culture

In some aspects, cells may be cultured for at least between about 10 days and about 40 days, for at least between about 15 days and about 35 days, for at least between about 15 days and 21 days, such as for at least about 15, 16, 17, 18, 19 or 21 days. In some aspects, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some aspects, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other aspects may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular aspects, cells are cultured in a cell culture system comprising a cell culture medium, for example in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular aspects of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain aspects cells are introduced into a culture system that features at least one substrate surface that is generally compatible with adherence of cells thereto, such that the plated cells can contact the said substrate surface, such aspects encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain aspects, the marker proteins used to identify and characterize the cells are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

### C. Pharmaceutical Compositions

In certain aspects, the compositions or agents for use in the methods, such as tolerogenic compositions described herein (e.g., tolerogenic nanocarriers), are suitably contained in a pharmaceutically acceptable carrier. In some aspects, the carrier is non-toxic, biocompatible and is selected so as not to detrimentally affect the biological activity of the agent. The agents in some aspects of the disclosure may be formulated into preparations for local delivery (i.e. to a specific location of the body, such as or other tissue) or systemic delivery, in solid, semi-solid, gel, liquid or gaseous forms such as tablets, capsules, powders, granules, ointments, solutions, depositories, inhalants and injections allowing for oral, parenteral or surgical administration.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### D. Other Agents

It is contemplated that other agents may be used in combination with certain aspects of the present aspects to improve the therapeutic efficacy of treatment. These additional agents include agents that stimulate generation of tolerogenic antigen presenting cells and/or formation of antigen-specific Tregs.

### EXAMPLES

The following examples are included to demonstrate certain aspects of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific aspects which are disclosed and still obtain a like or similar result without departing from the spirit and scope of the invention.

### Example 1—Robust Tolerogenic Dendritic Cells Via a Push/Pull Pairing of Toll-Like-Receptor Agonists and Immunomodulators Reduces Clinical Signs of EAE

A failure of central immune tolerance typically driven by autoantigen specific T regulatory (Treg) cells is the primary cause of many autoimmune diseases. While the specifics of this failure vary between diseases, restoration of a proper autoantigen specific response holds promise as a highly effective, long-term therapy for a wide variety of autoimmune diseases. Generating autoantigen specific Tregs, however, remains a challenge due to the non-specific nature of most tolerizing agents and the complexities of generating Tregs in vivo. In this study, a new push/pull method for inducing antigen-specific Treg responses is demonstrated via induction of tolerogenic dendritic cells (tolDC, PD-L1+, CD80− CD11c+). In particular, a combination of three immunosuppressive agents was identified, dexsamethasone, simvastatin and SC-514, which when used in combination with TLR agonists induces an active tolDC phenotype. When the tolerogenic combination was packaged into a liposome with a model antigen such as ovalbumin (OVA), these tolDCs induced differentiation of OVA specific Tregs both ex vivo and in vivo. The tolerizing potential of the combination was examined in an EAE disease model. Tolerogenic liposomes were loaded with myelin oligodendrocyte glycoprotein (MOG) peptide (a peptide on mouse myelin sheath cells) and used in EAE autoimmune mouse model to reduce clinical signs of EAE. Given the antigen specificity and ease of antigen substitution of this technique, these studies present an attractive autoimmune therapy.

By incorporating a TLR agonist, immunomodulator and antigen into a single nanoparticle, it was hypothesized that more effective tolDC responses could be generated, which in turn would generate both more effective Tregs and better autoimmune suppression (FIG. 1A). While TLR agonists are widely used in vaccine research, using TLR agonists or inhibitors to trigger tolerance is also documented in the literature. Small doses of lipopolysaccharide (LPS) have been shown to desensitize cells to other TLR agonists and lead to tolerance and increase tolerogenic cytokines such as IL-10, but this is dangerous as LPS can also trigger strong inflammatory responses. (17) Another potential benefit of TLR activation for tolDCs is that optimal Treg responses is achieved from suboptimal, not non-existent, T cell co-stimulation. (18) This means that tolDCs need to generate a small amount of CD28 or CD40 expression in combination with PD-L1. Such positive co-stimulation is best achieved via TLR activation. (19) Furthermore, exposure to TLR agonists leads to DC maturation, leading to longer lived phenotypes and upregulation of survival signals. (20) The hypothesis was that exposure to the appropriate combination of TLR agonist and immunomodulators would generate tolDCs that are long-lived and which actively and strongly stimulate Treg differentiation. A challenge in this study was to find a combination of TLR agonist and immunomodulators that generated this tolDC phenotype.

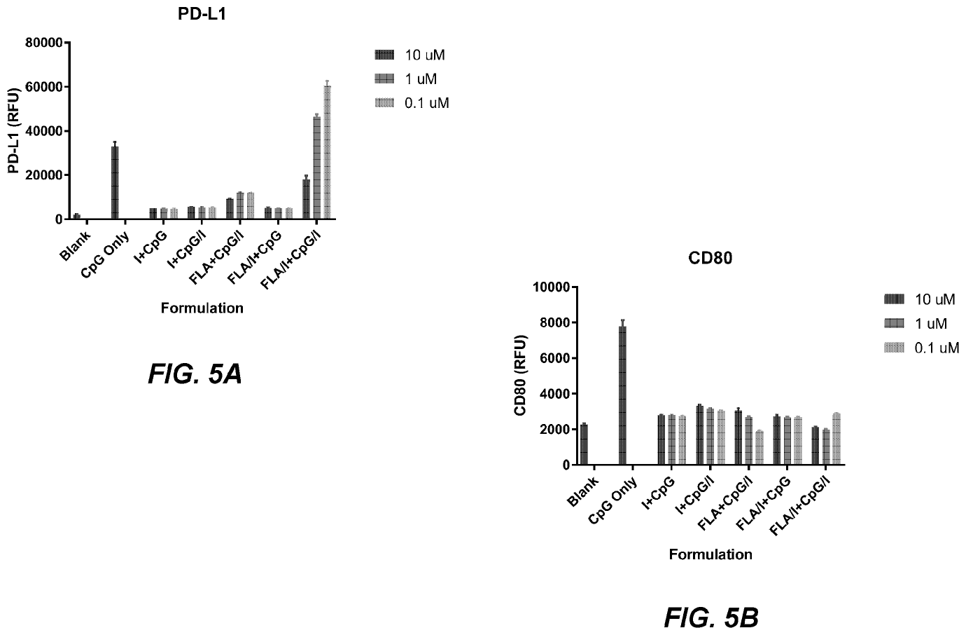

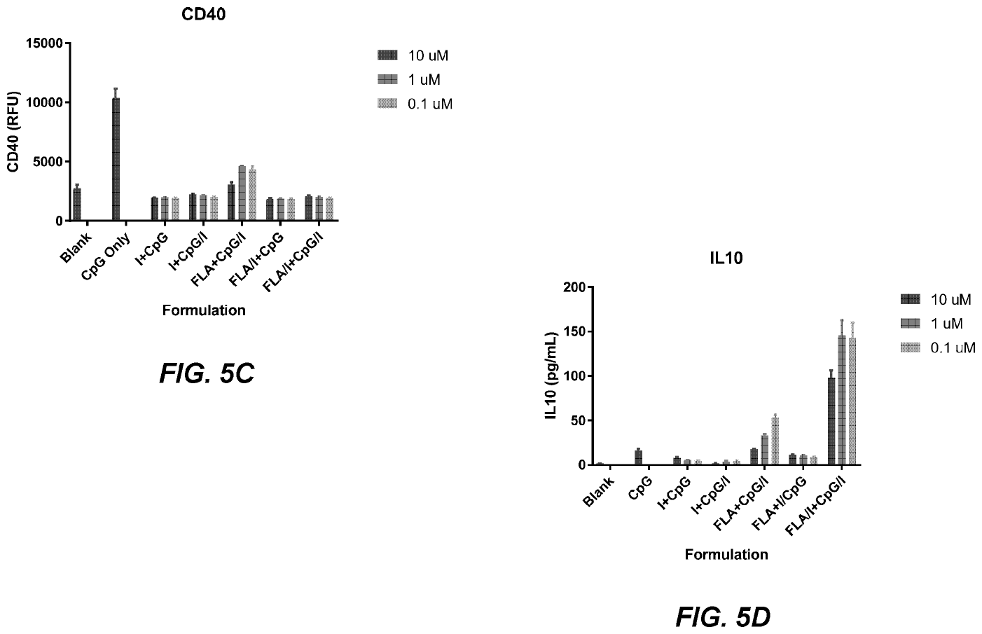

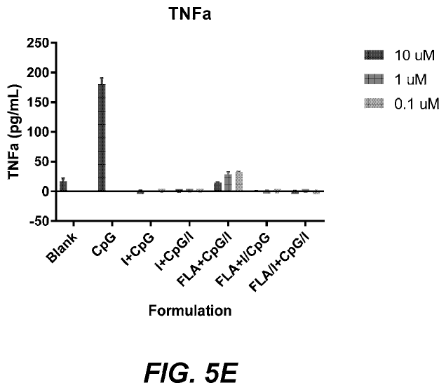

First, a library of TLR agonists and immunomodulators was obtained to test in vitro with APC cell lines and BMDCs to optimize this TLR agonist/immunomodulator combination. After validating these combinations of TLR agonists and modulators do indeed increase tolDC phenotypes, a library of TLR agonists and immunomodulators was purchased based on previous literature (FIGS. 5A-5E, Table 1).

TABLE 1
List of immunomodulators and TLR agonists tested

| # | Inhibitor Name | Type | MW | | Final Conc | | Vendor |
|---|---|---|---|---|---|---|---|
| 1 | Dexamethasone | cholesterol like | 392.46 | | 1 | μM | Med Chem Express |
| 2 | rapamycin | Cyclic lipid | 914.17 | | 10 | nM | Med Chem Express |
| 3 | Ridaforolimus | Cyclic lipid | 990.21 | | 10 | nM | Selleck Chem |
| 4 | acetylsalicylic acid | Small molecule | 180.16 | | 1 | mM | Sigma |
| 5 | ly294002 | small molecule | 343.8 | | 1 | μM | Med Chem Express |
| 6 | simvastatin | small molecule | 418.57 | | 10 | ug/mL | Med Chem Express |
| 7 | IRF3-siRNA | siRNA | 13300 | | 10 | nM | Sigma |
| 8 | 3′-Sialyl Lewis X | Sugar | 820.74 | | 10 | ug/mL | Carbo Synth |
| 9 | Cholecalciferol (Vitamin D3) | Cholesterol-like | 384.46 | | 1 | μM | Sigma |
| 10 | Vasoactive intestinal peptide | Peptide | 3325 | | 10 | nM | Sigma |
| 11 | Lifitegrast | small molecule | 615.5 | | 100 | nM | Camen Chemical |
| 12 | TSLP | protein | 15.4 | kDA | 10 | ng/mL | SinoBiologicals |
| 13 | minocycline | antiboiotic | 493.94 | | 5 | μM | Sigma |
| 14 | Hydroxychloroquine | small molecule | 433 | | 1 | μM | Med Chem Express |
| 15 | Ligustilide | small molecule | 190.4 | | 20 | μM | Med Chem Express |
| 16 | Sapanisertib | small molecule | | | 10 | nM | Selleck Chem |
| 17 | HGF mouse protein | protein | 79.3 | kDa | 100 | ng/mL | Sino Biological |
| 18 | Retinoic acid | Small Molecule | 300.44 | | 1 | μM | Med Chem Express |
| 19 | BX-795 | Small molecule | 591.47 | | 1 | μM | Sigma |
| 20 | SB203580 | small molecule | 377.44 | | 10 | μM | Invivogen |
| 21 | SP600125 | small molecule | 220.2 | | 10 | μM | Invivogen |
| 22 | Curcumin | small molecule | 368.38 | | 25 | μM | Sigma |
| 23 | Bee venom PLA2 | enzyme | | | 0.1 | ug/mL | Sigma |

| | | | | | | |
|---|---|---|---|---|---|---|
| 24 | PI103 | small molecule | 348.36 | 100 | nM | abcam |
| 25 | Parthenolide | small molecule | 248.3 | 100 | nM | abcam |
| 26 | SC 514 | small molecule | 224.3 | 100 | nM | Tocris |

| Agonist Name | TLR | MW | | Concentration | | Vendor |
|---|---|---|---|---|---|---|
| LTA (Lipoteichoic acid BS) | TLR2 | 4-12 | kDa | 10 | ug/mL | Invivogen |
| LPS (Lipopolysaccaride EK) | TLR4 | 50-100 | kDa | 0.1 | ug/mL | Invivogen |
| FLA (Flagellin BS) | TLR5 | 32 | kDa | 0.1 | ug/mL | Invivogen |
| R848 | TLR7 | 350.8 | Da | 1 | ug/mL | Invivogen |
| CpG (ODN 1826) | TLR9 | 6383 | Da | 1 | μM | Invivogen |
| BF (bacteria fragilis, | TLR2 | n/a | | 10\hat{ }6 cells | | Invivogen |
| heat killed) | | | | per mL | | |
| HSP60 (Heat Shock Protein 60) | TLR4 | 57-59 | kDa | 1 | ng/mL | NovusBio |

Mouse bone marrow derived dendritic cells (BMDCs) or RAW Blue cells were dosed with varying concentrations and combinations of TLR agonists/modulators (Table 1). To test the potential for a tolerogenic response, cells were challenged after 16 h with the TLR9 agonist CpG (ODN 1826). The levels of PD-L1, CD80, CD40, IL-10 and IL-6 were measured for BMDCs and the levels of NF-κB activity was measured using SEAP reporter assay on the RAW Blue cells. A tolerance score was calculated by assessing the relative contributions of tolerizing activity (PD-L1, IL-10) to inflammatory activity (CD80, CD40, IL-6 and NF-κB) (Table 2, see methods).

TABLE 2
Preliminary screen of tolerizing and agonist combinations.

| First Agonist | Second Agonist | Inhibitor | Tolerance Score |
|---|---|---|---|
| FLA | CpG | 6 | 38.9 |
| None | CpG | 6 | 33.5 |
| BF | CpG | 6 | 23.4 |
| FLA | CpG | 1 | 23.1 |
| CpG | FLA | 6 | 22.7 |
| HSP60 | CpG | 6 | 20.6 |
| FLA | CpG | 26 | 15.0 |
| HSP60 | CpG | 5 | 11.7 |
| None | CpG | 13 | 11.1 |
| CpG | FLA | 14 | 11.0 |
| CpG | FLA | 8 | 10.8 |
| None | CpG | 12 | 10.1 |
| FLA | CpG | 11 | 10.0 |
| HSP60 | CpG | 12 | 9.6 |
| None | CpG | 11 | 9.6 |
| HSP60 | CpG | 14 | 9.4 |
| None | CpG | 5 | 8.2 |
| CpG | FLA | 1 | 8.1 |
| CpG | FLA | 5 | 8.0 |
| CpG | FLA | 5 | 7.9 |
| None | CpG | 1 | 7.5 |
| None | CpG | No Inhibitor | 7.3 |
| HSP60 | CpG | No Inhibitor | 7.1 |
| CpG | FLA | No Inhibitor | 6.9 |
| FLA | CpG | No Inhibitor | 6.9 |
| None | CpG | 14 | 6.7 |

Three compounds were taken forward from this preliminary screen for further testing based on their tolerizing effects: dexamethasone (1), simvastatin (6), and SC-514 (26). While dexamethasone and simvastatin have previous clinical data in autoimmune treatment, SC-514 (a NF-κB inhibitor) has almost no data and has yet to be analyzed clinically. (21-23) The inventors identified that this cocktail of inhibitors synergistically improved tolDC phenotype when combined with the TLR agonist Flagellin (FLA) and then subsequently challenged a combination of TLR agonist CpG (ODN 1826) and the same cocktail of inhibitors (FIGS. 1B and 1C).

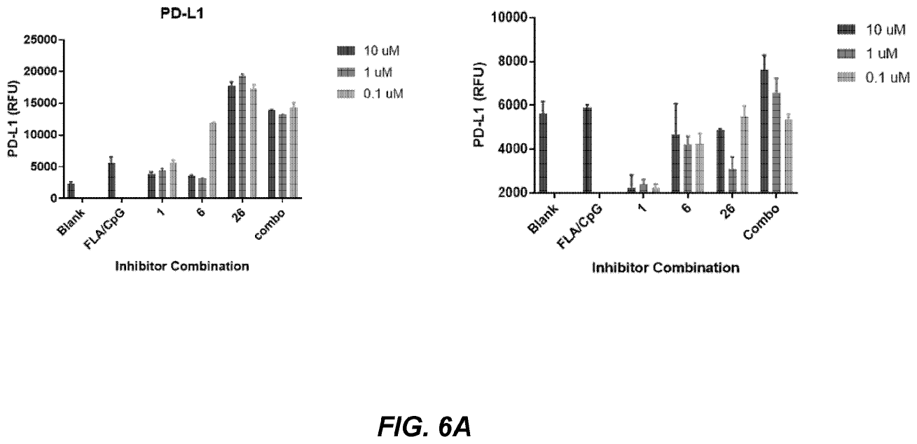

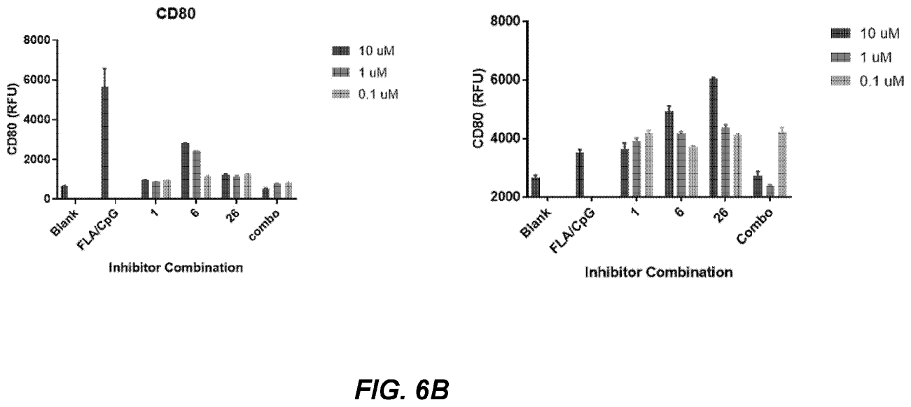

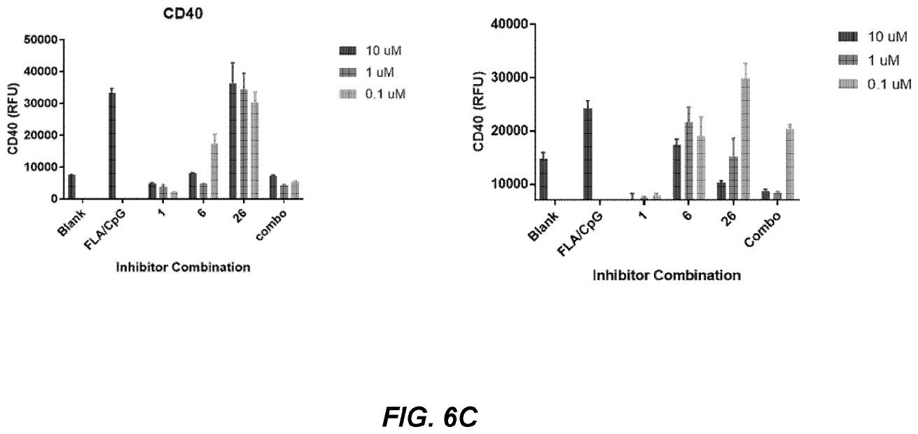

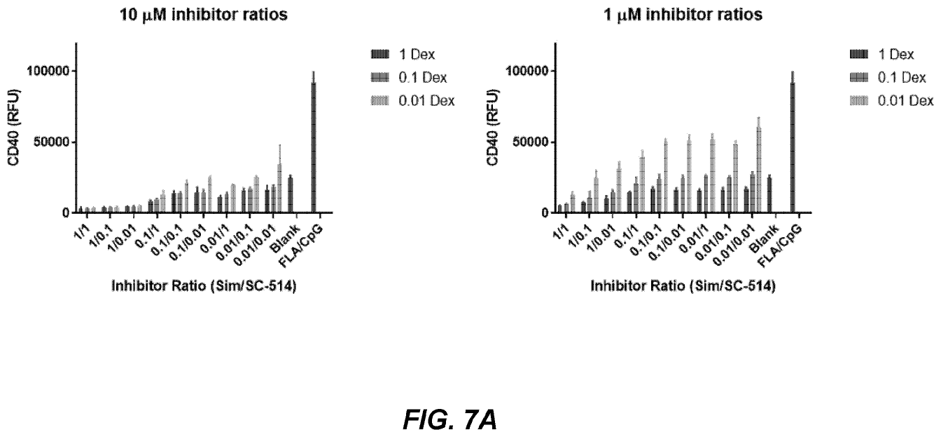

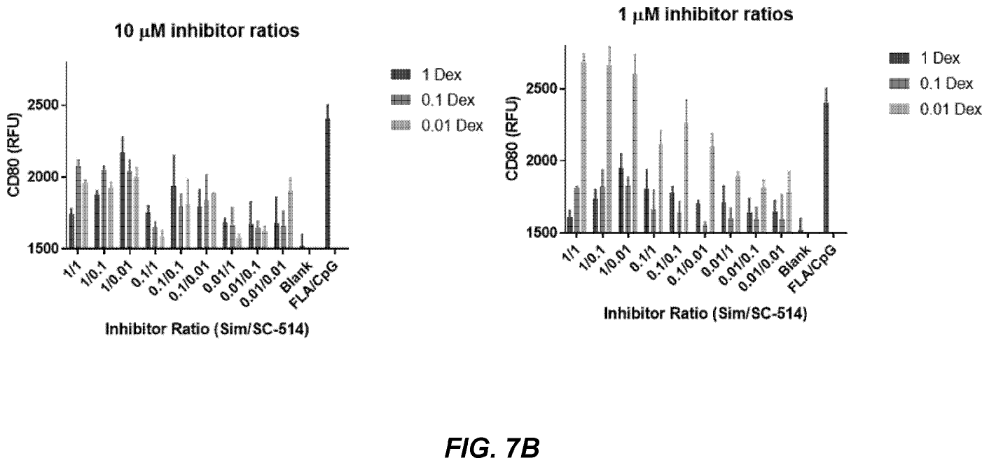

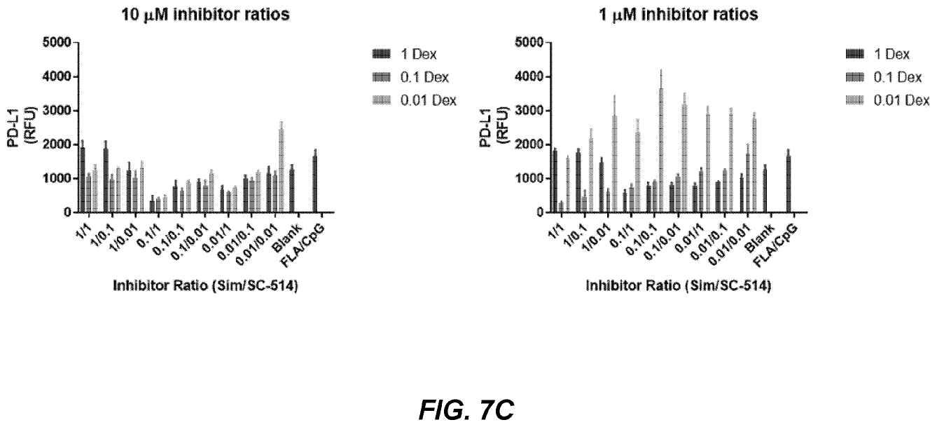

The dosing strategy was optimized and it was determined that adding the inhibitors with FLA on day 1, washing and then adding CpG with inhibitors on day 2 resulted in the highest tolerance score (FIGS. 5A-5E). This formulation and dosing regimen also led to the greatest increase in tolDC phenotype in BMDCs. Similar tolDC phenotype markers were observed in DCs extracted from mouse spleens and innate immune cell lines such as DC2.4 and RAW 264.2 (FIGS. 1D and 1E, FIGS. 6A-6C). During the investigation of this study, many ratios and concentrations were tested of all three tolerogenic drugs and TLR agonists in BMDCs, but it was found that the most effective formulation ranged between 10-1 μM total inhibitor concentration (of all three drugs at a 1:1:1 ratio) combined with either 0.1 μg/mL of FLA or 0.5 μM of CpG (FIGS. 7A-7C). For clarity, formulations with TLR agonists containing 1:1:1 Dex:Sim:SC-514 are denoted as (TLR+I), formulations containing just TLR agonists as (TLR), and formulations containing just inhibitor as (I). Note that the treatment regimen of dosing with FLA-containing formulations on day 1 followed by CpG-containing formulations on day 2 was used in all the experiments of this study unless noted otherwise.

It was hypothesized that the inclusion of TLR agonists with immunomodulators would increase tolDC viability and lifetime in addition to generating stronger tolDC phenotypes. To test this hypothesis, BMDCs were treated in a similar fashion as the previous experiment with a dose regiment of both concentration of TLR+I and time (FIGS. 1D and 1E). After the addition of the final TLR+I, cells were incubated for 24 hrs and tested for IL-10 and cytotoxicity using an MTT assay on day 1. Similarly treated cells were washed on day 3, challenged for 24 hrs with 0.5 uM of CpG and tested for IL-10 secretion and cytotoxicity on day 4. This procedure was subsequently repeated on day 6, 9 and 13. The results show that BMDC viability is low for inhibitor treated cells on day 1, but rises over time when compared to PBS control cells (FIG. 1F). BMDCs treated with both inhibitors and TLR agonists (TLR+I) had greater viability and longevity when compared to BMDCs treated with just inhibitors and no agonists (I). TLR+I treated cells also had an increase in IL-10 secretion, indicating that these cells were more actively tolerizing for longer than inhibitor only group.

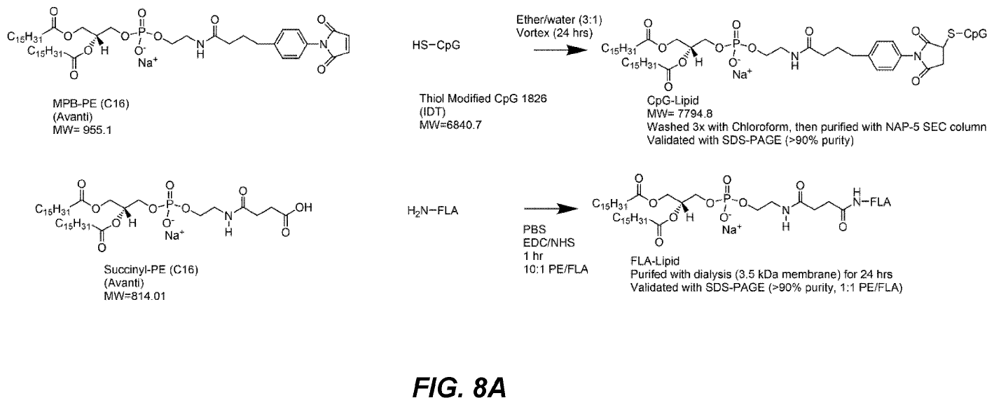

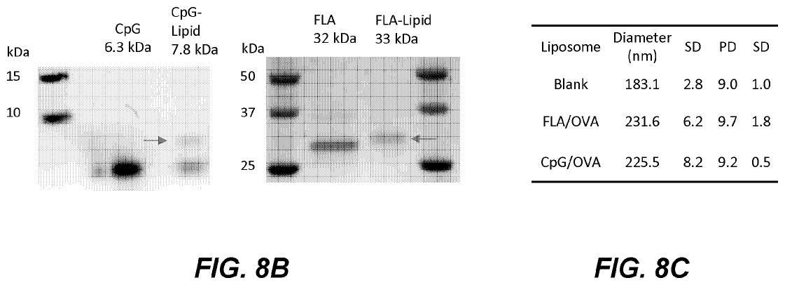

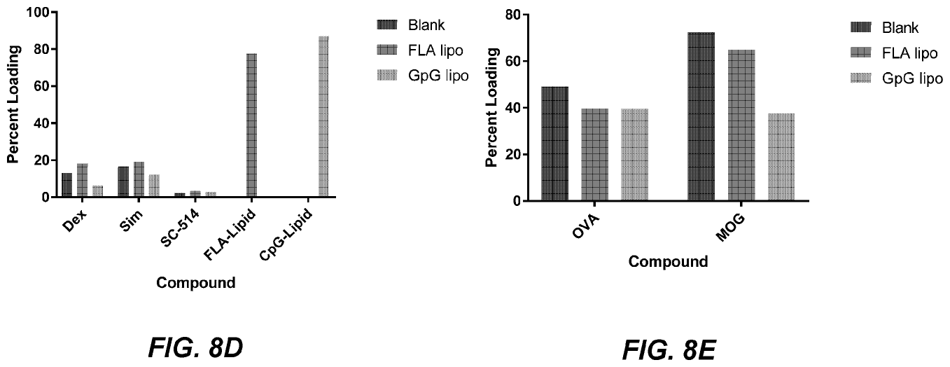

After optimizing the TLR agonist-inhibitor formulation, the inventors next sought to incorporate this formulation in an appropriate nanocarrier. A nanocarrier was required for any in vivo study for two reasons: (1) in order to ensure that all components of the inhibitor formulation were co-delivered and (2) provide antigen specificity. (24) Liposomes were chosen as nanocarriers as liposomes provide good passive targeting of innate immune cells and have been widely used in the clinic for other diseases. (25) Also, liposomes can readily be formulated with all three tolerance drugs (Dex, Sim and SC-514, FIGS. 8A-8E). However, the TLR agonists FLA and CpG are too hydrophilic to reliably be incorporated into liposomes. Furthermore, it is desirable that the TLR agonists be displayed on the surface of a liposome to ensure the agonists stimulate TLRs during phagocytosis. To achieve this, the inventors conjugated FLA and CpG with lipid (C14) tails using established chemistries, purified them using HPLC and confirmed that FLA and CpG-lipid conjugates are incorporated into 200 nm diameter liposomes at >95% efficiency (FIGS. 8A-8E). (26) Cells and mice were both applied using the same protocols as previous free drug formulations, receiving FLA/inhibitor liposomes first, then CpG/inhibitor liposomes the following day. For groups with no TLR agonist, cells/mice were treated on consecutive days with the same liposome formulation (with or without inhibitors). For clarity, groups treated with liposomes bearing TLR agonists and inhibitors were denoted as Lipo^{TLR+I}, groups treated with inhibitor loaded liposomes with no TLR agonist as Lipo^{I} and groups treated with TLR agonist bearing liposomes only as Lipo^{TLR}.

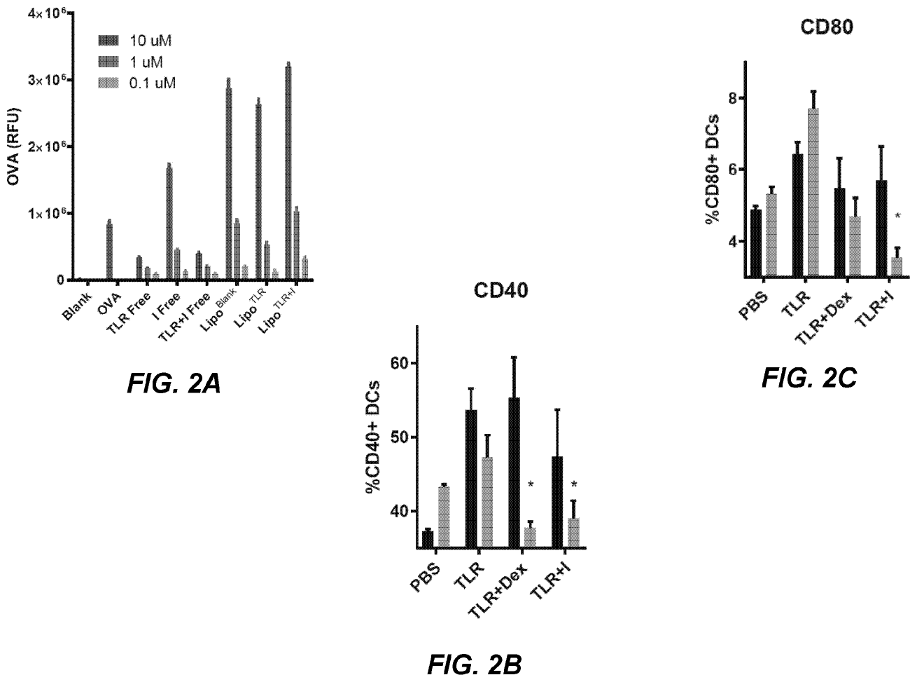

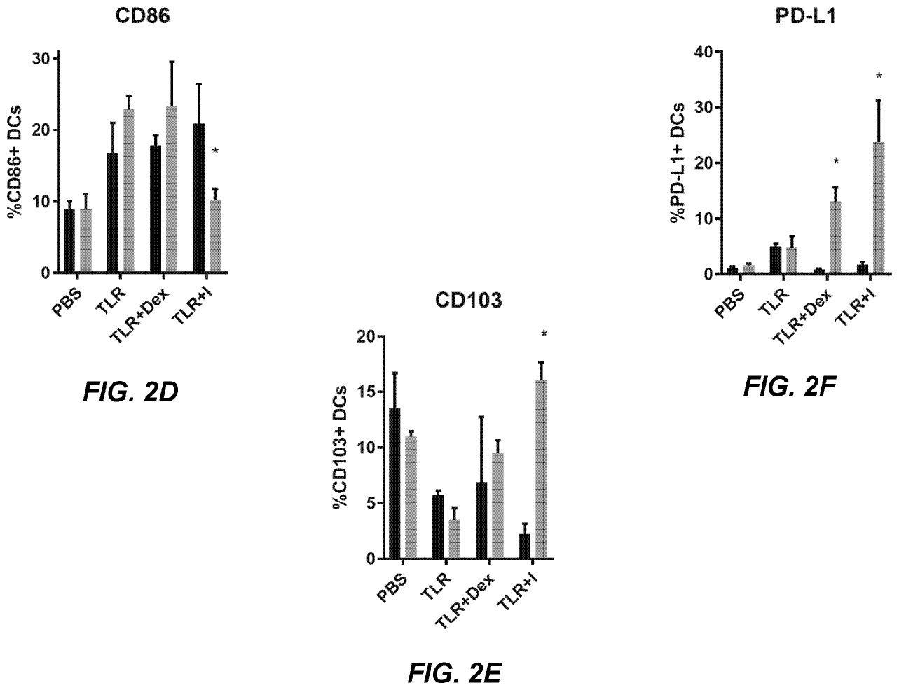

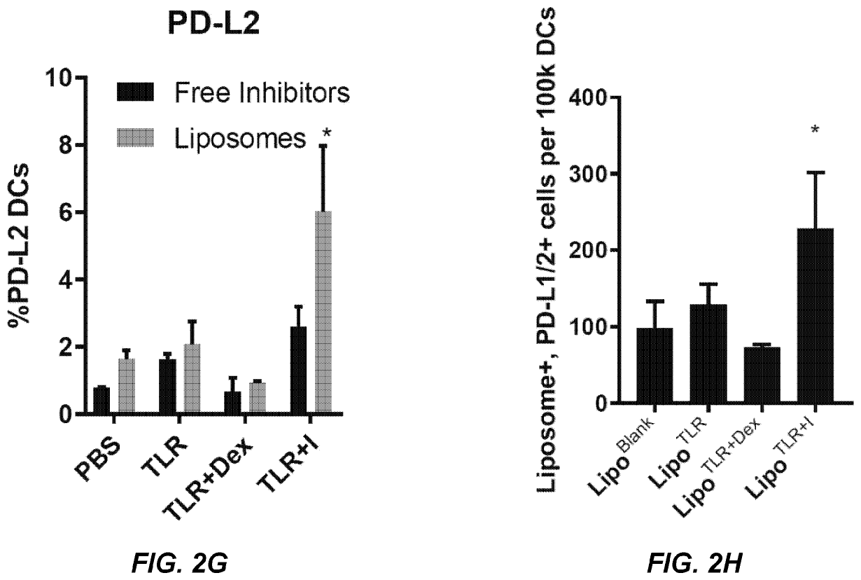

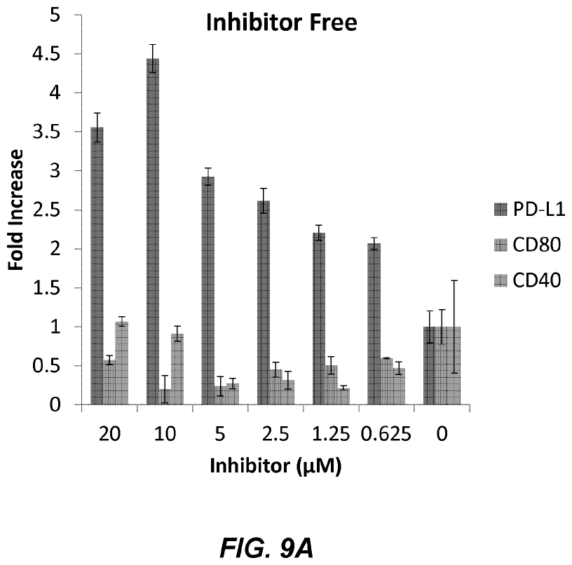

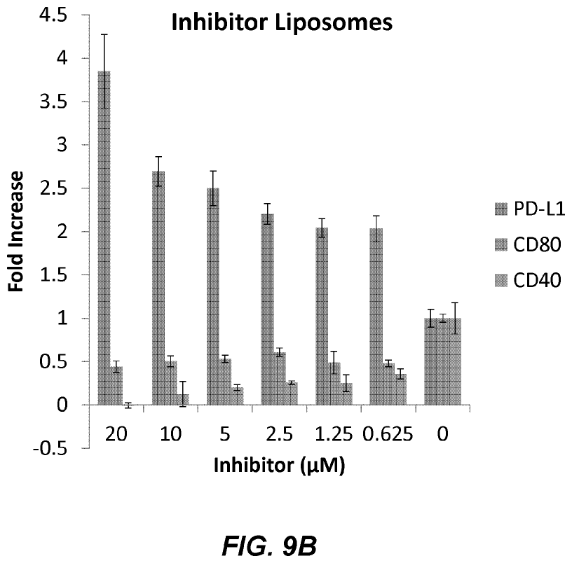

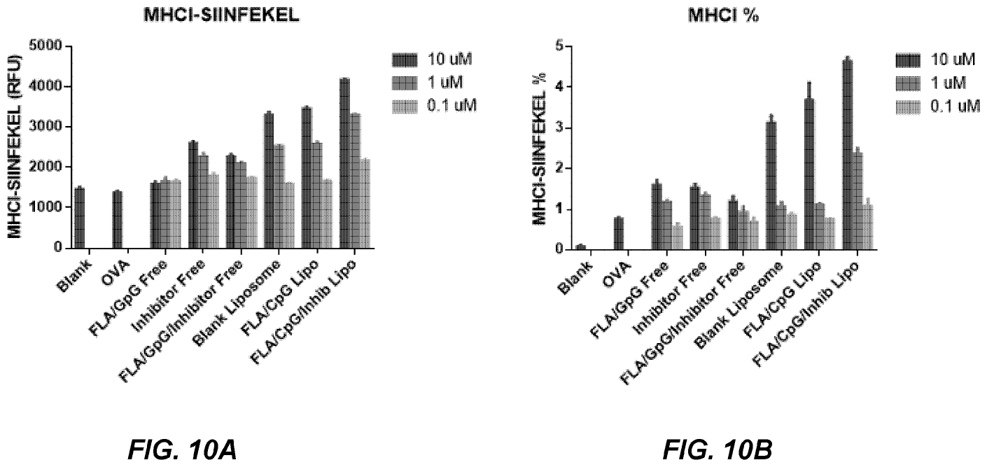

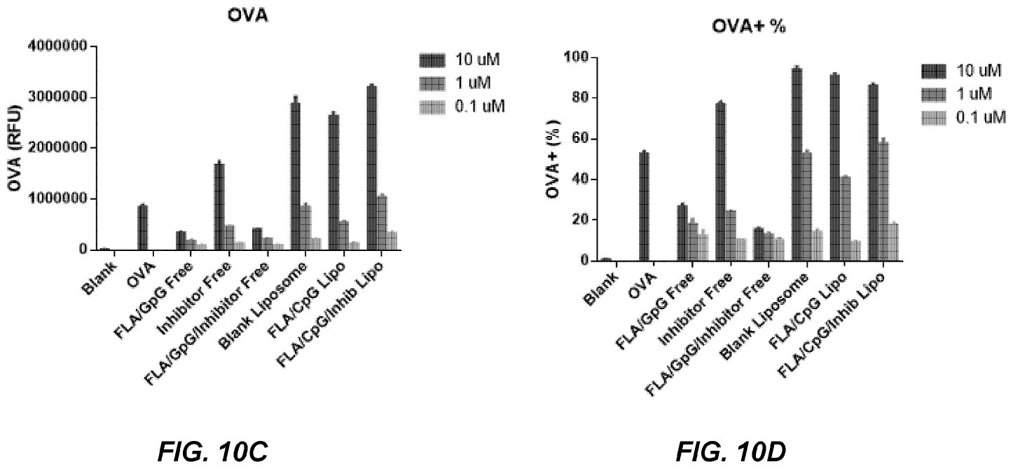

After validating that the tolerogenic formulations contained the expected levels of compounds, the inventors sought to show that these formulations induce antigen specific tolDCs both in vitro and in vivo. First it was validated that Lipo^{TLR+I} and Lipo^{TLR} formulations generate similar tolerogenic DC phenotypes as free formulations of TLR/inhibitors in vitro using BMDCs (FIGS. 9A-9B). Next, using the fluorescently labeled model antigen Ovalbumin (OVA), the inventors observed OVA uptake in BMDCs with or without the inhibitor formulations both in free and liposomal formulations (FIG. 2A, FIGS. 10A-10D). There was no decrease in OVA uptake for Lipo^{TLR+I} compared to blank liposomes (Lipo^{Blank}). An increase in the OVA major MHC I epitope presentation was observed for Lipo^{TLR+I} groups compared to Lipo^{Blank} (FIGS. 10A-10D). Interestingly, there was also a decrease in OVA uptake and MHCI epitope presentation for BMDCs treated with free tolerogenic inhibitors, indicating that a liposomal formulation may enhance effective antigen presentation in vivo.

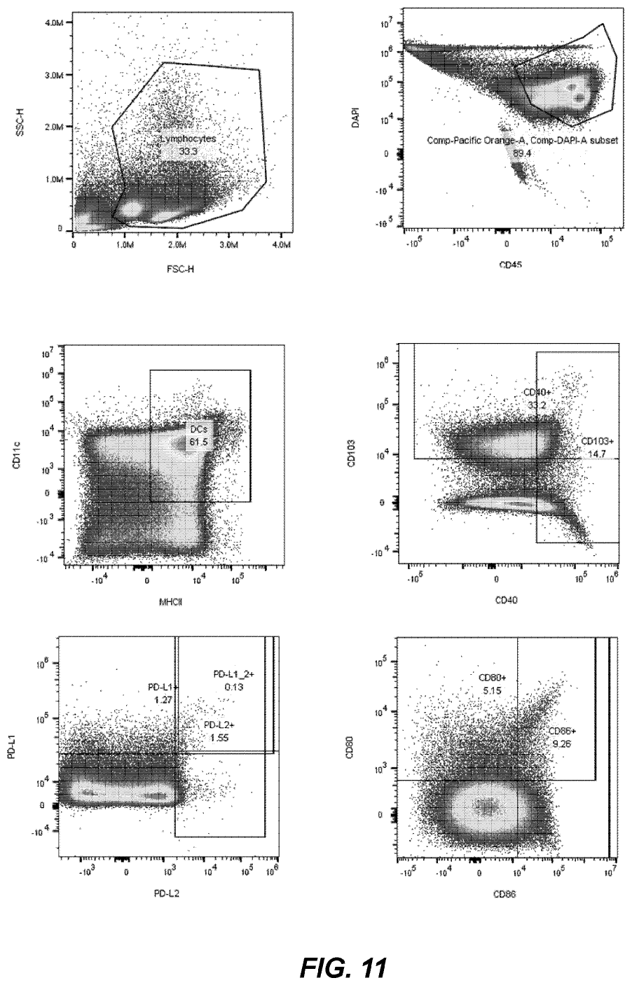

Next, the inventors sought to demonstrate that the tolerogenic liposomes can generate tolDC populations in vivo. Mice were injected with either free or liposomal formulations of all three inhibitors (TLR+I), dexsamethasone with TLR agonists (TLR+Dex), with only TLR agonists (TLR) or blank PBS controls (free PBS or blank liposome). Dex only containing formulations were chosen as a control because dex has been used in previous studies for tolerance, and the inventors wanted to observe the effects of the drug combination compared to dex. (27) All mouse groups were injected with FLA formulations first, then 24 hrs later injected with CpG formulations (except PBS, which was injected with just PBS on both days) then sacrificed and lymph nodes analyzed 24 hrs after CpG injection. This dosing regimen mirrored the dosing strategy optimized in vitro (Table 2). All formulations contained 100 μg of OVA in the second injection. DCs from mouse lymph nodes were analyzed via spectral flow cytometry for DC inflammatory markers CD40, CD80 and CD86 and DC tolerogenic markers such as CD103, PD-L1 and PD-L2 (FIGS. 2B-2G, FIG. 11 for gating strategy). A significant increase was observed in all tolerogenic markers and a decrease in all inflammatory markers in mice treated with the combination of tolerogenic liposomes when compared to the formulations without inhibitor. To monitor which DCs phagocytose the liposomes, all liposomes were labeled with a DiD dye. Tracking this dye, it was determined that Lipo^{TLR+I} significantly increased the number of liposome+, PD-L1/2+ tolDCs (FIG. 2H).

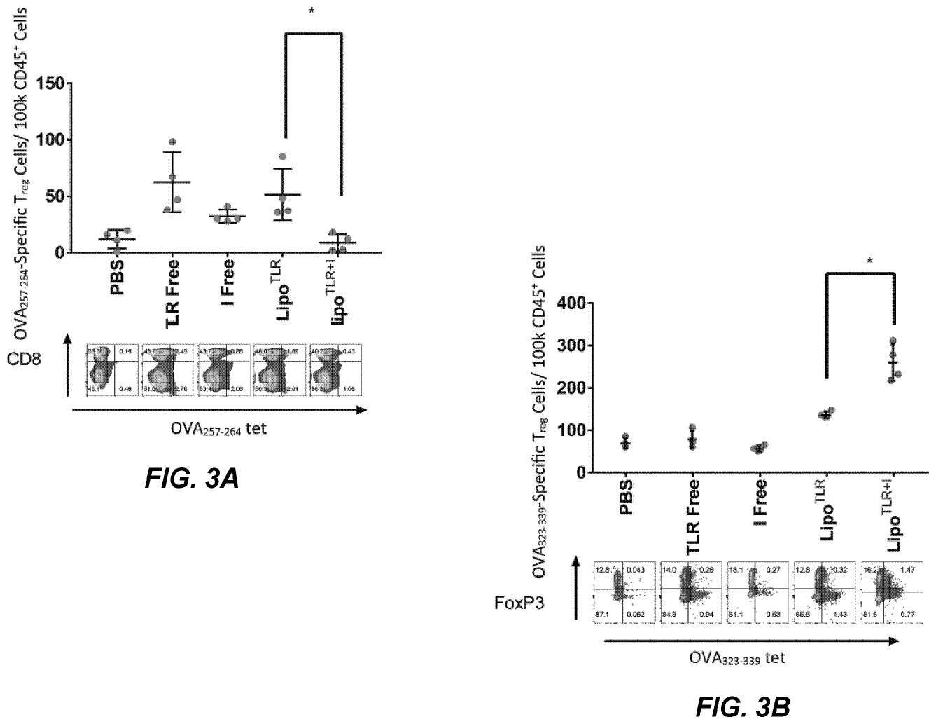

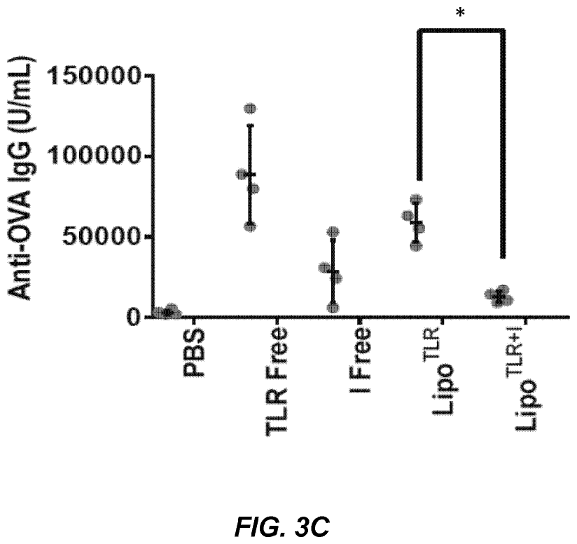

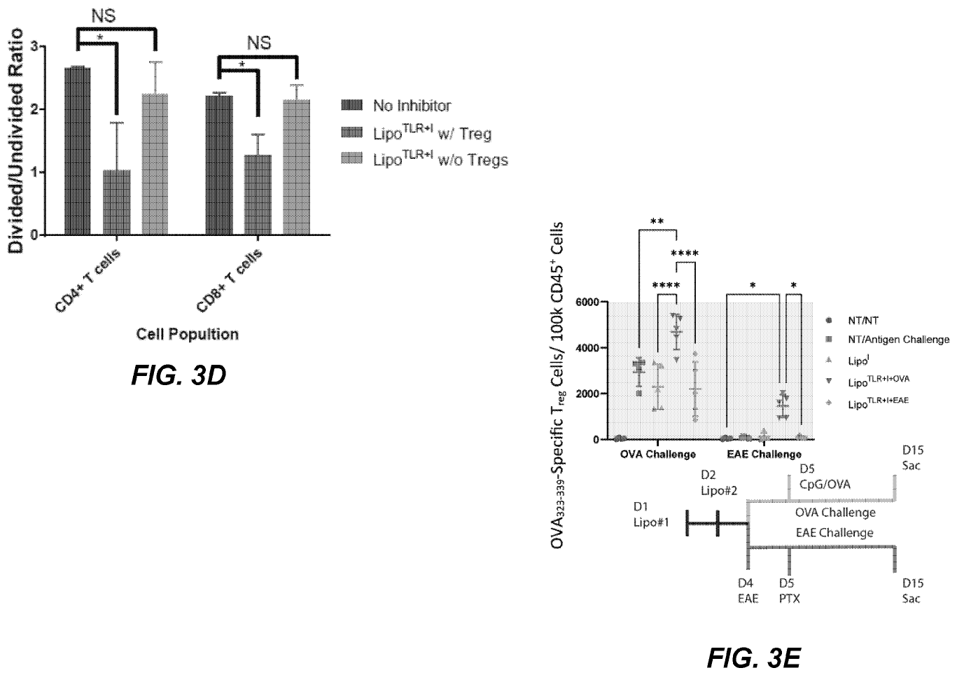

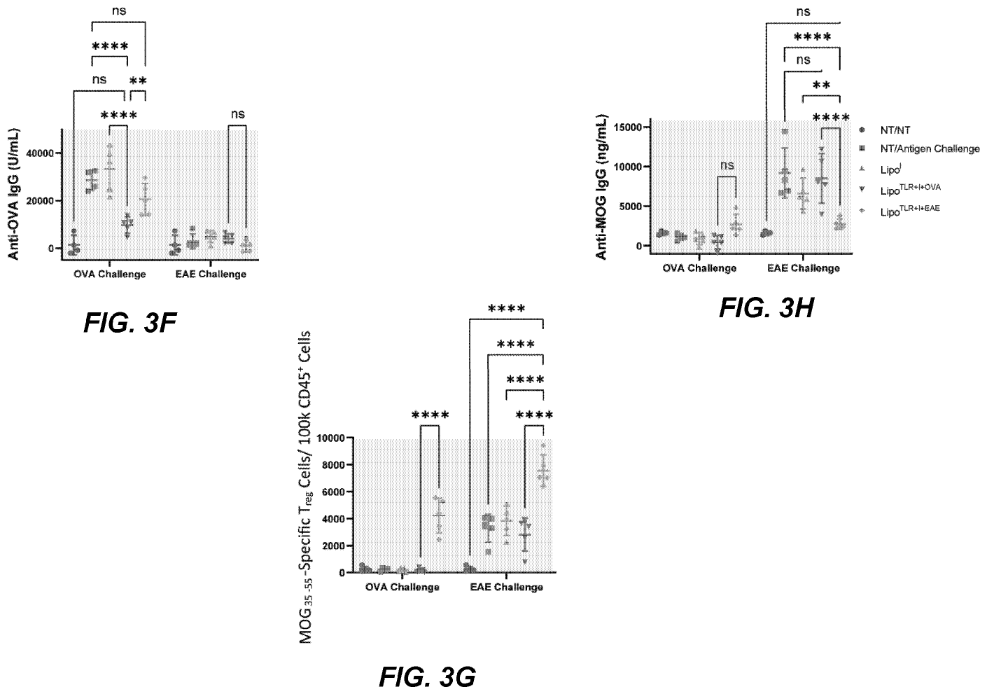

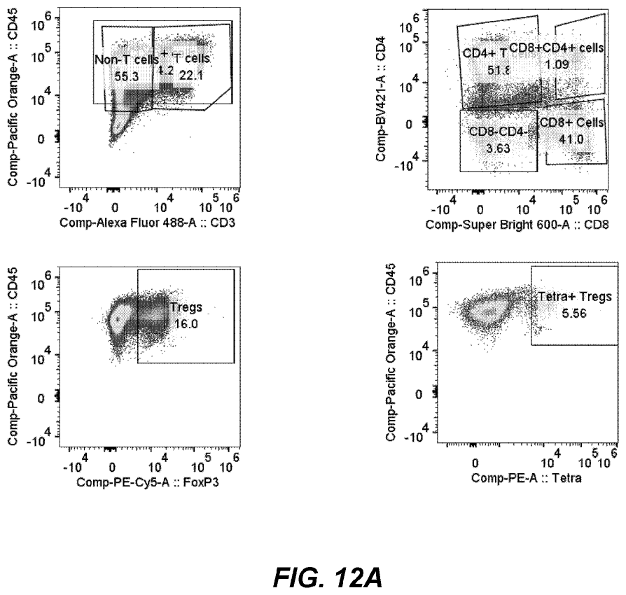

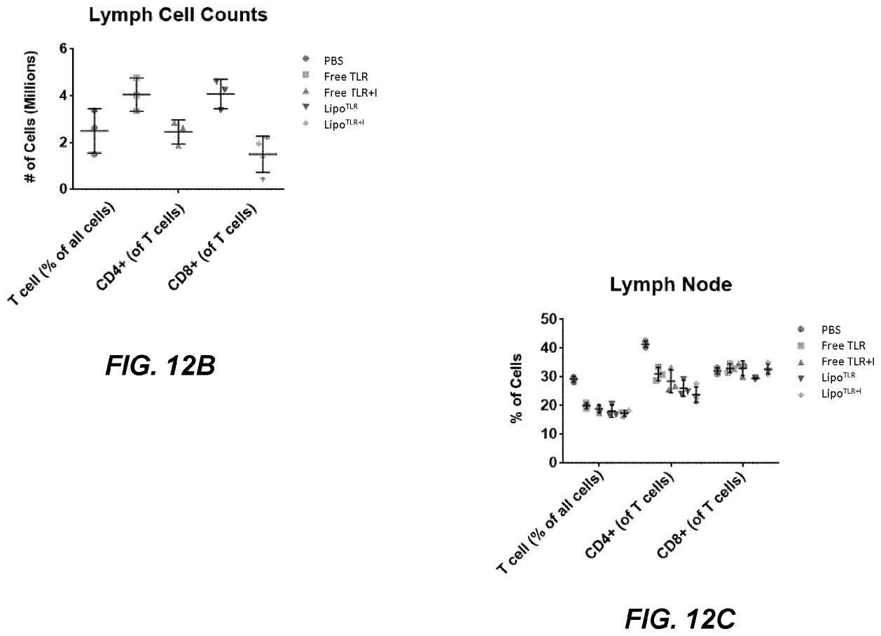

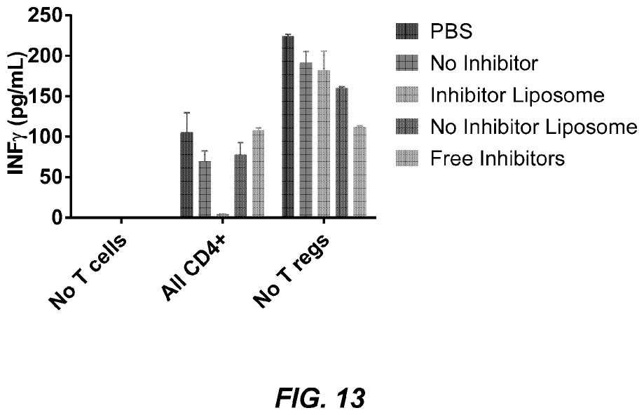

After demonstrating that Lipo^{TLR+I} generated tolDCs, the next goal was to show that these tolDCs reduce antigen specific immunity and generate antigen specific Tregs. Similar to the previous experiment, mice were injected with OVA+Lipo^{TLR+I}, OVA+Lipo^{TLR} or the same mixture of molecules not in liposomes. 10 days after the second injection, mice were sacrificed and serum analyzed for OVA specific IgGs, showing a significant decrease in IgGs for Lipo^{TLR+I} (FIG. 3A). Lymphocytes were also stained with a tetramer for the major MHCI epitope of OVA and analyzed via flow. While a change in the overall CD8 or CD4 ratios was not observed, there was a decrease in CD8⁺, MHCI-epitope tetramer positive T cells in tolerogenic liposome treated mice (FIG. 3B, FIGS. 12A-12C). Furthermore, there was an increase in MHCII-tetramer positive Treg cells for Lipo^{TLR+I} group (FIG. 3C). This result indicated that the liposomal treatment did not alter global T cell populations, but rather antigen specific T cells were selectively removed through OVA antigen specific Tregs. Finally, the inventors wanted to evaluate the functionality of the Tregs—specifically how well they suppress antigen specific T cell function. To do this, spleens were extracted, splenocytes were incubated with BMDCs presenting OVA and T cell proliferation and cytokine release after 48 hrs was observed. A significant decrease was observed in T cell proliferation in the spleens of mice administered Lipo^{TLR+I} (FIG. 3D). This suppression was recovered when generated Tregs were removed via a magnetic cell sorter, further indicating that Tregs were responsible for T cell suppression. A similar trend was also seen in the IL-2 and IFN-γ release (FIG. 3E, FIG. 13).

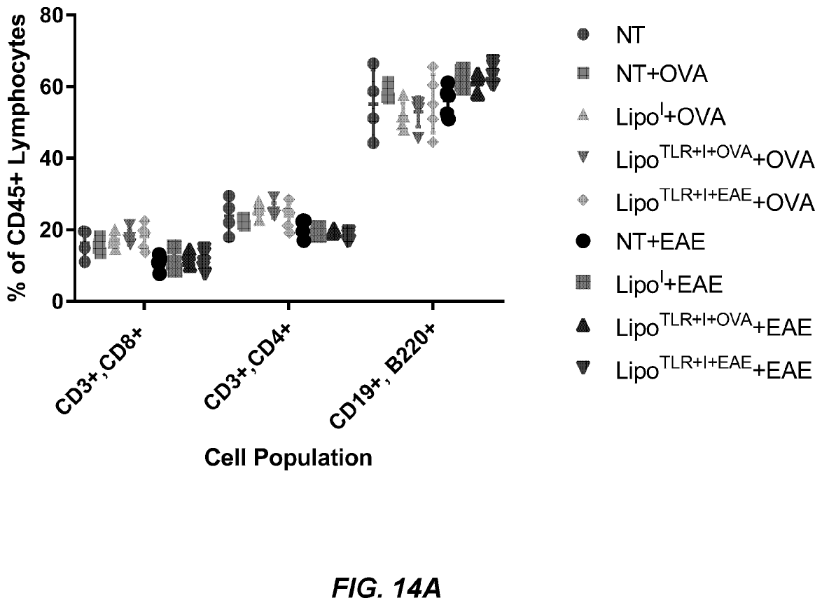

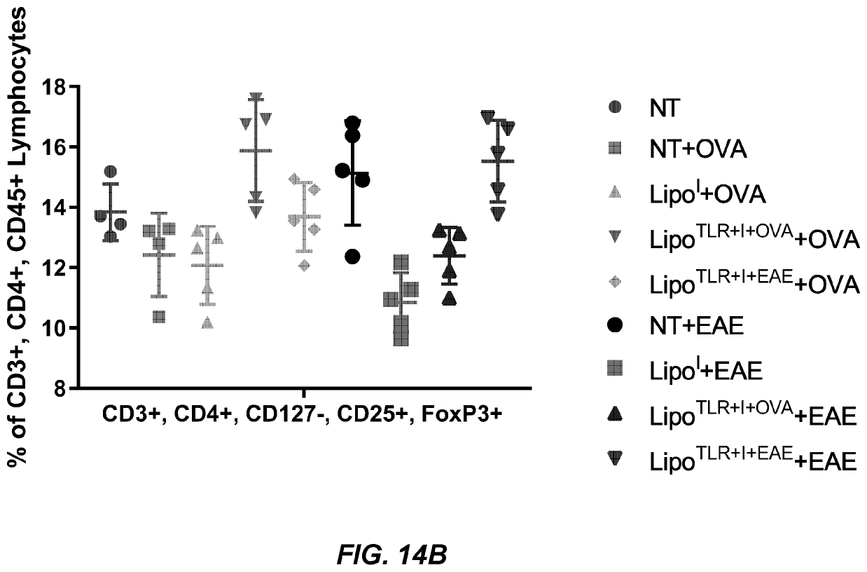

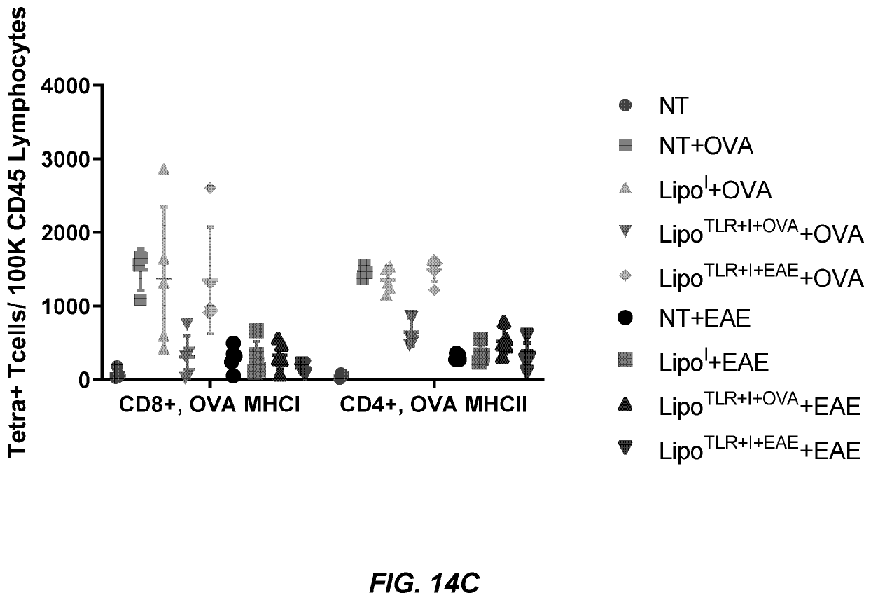

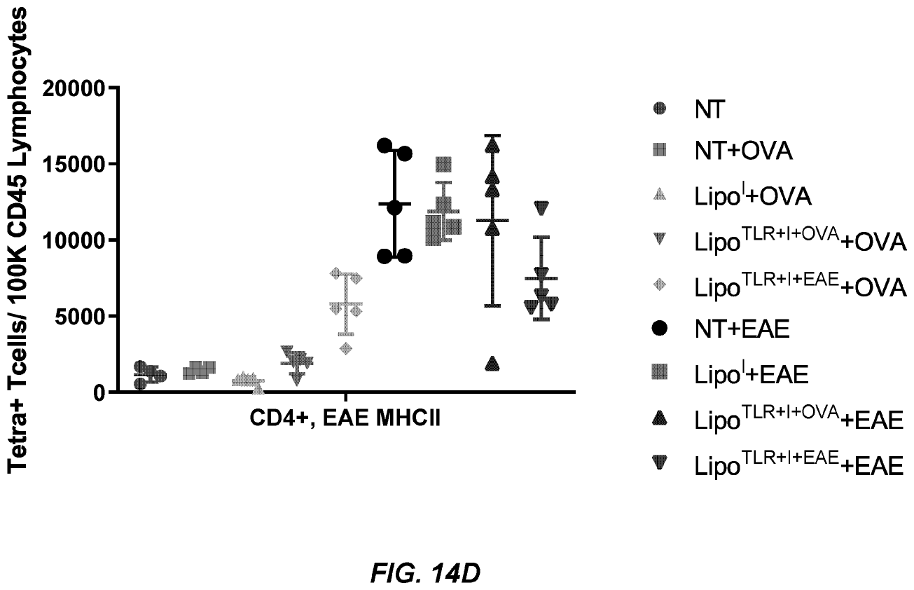

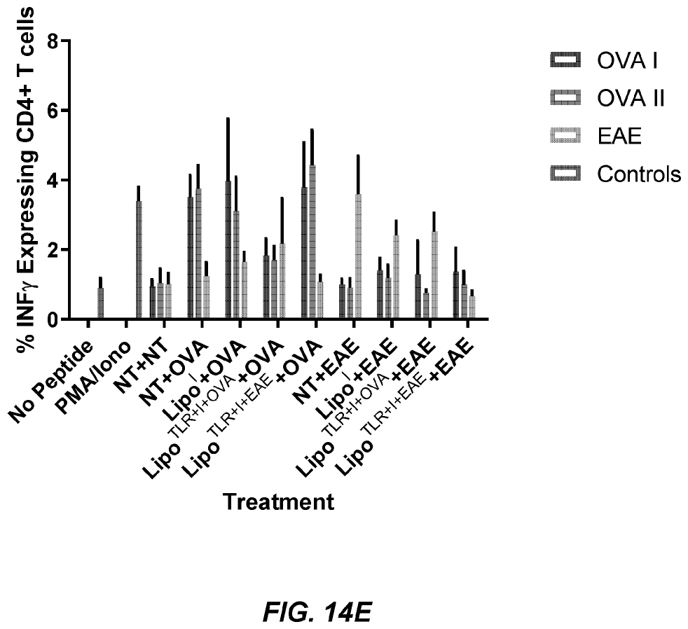

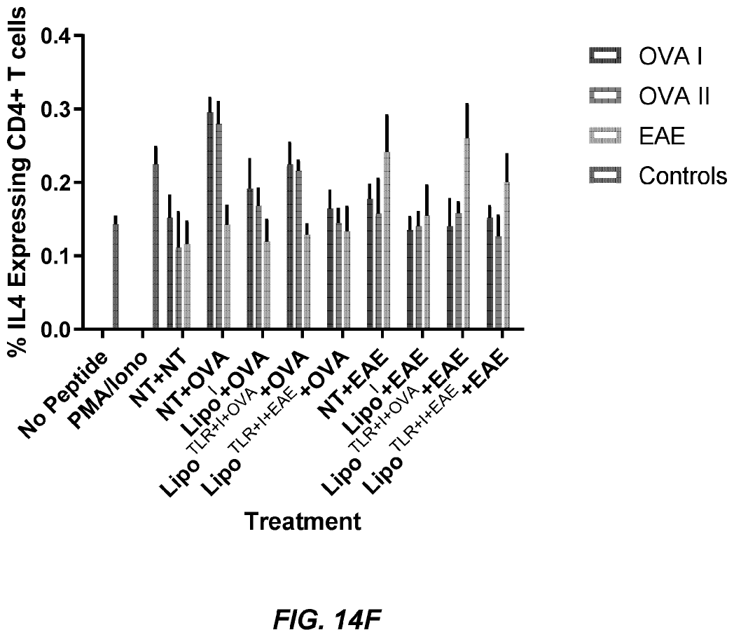

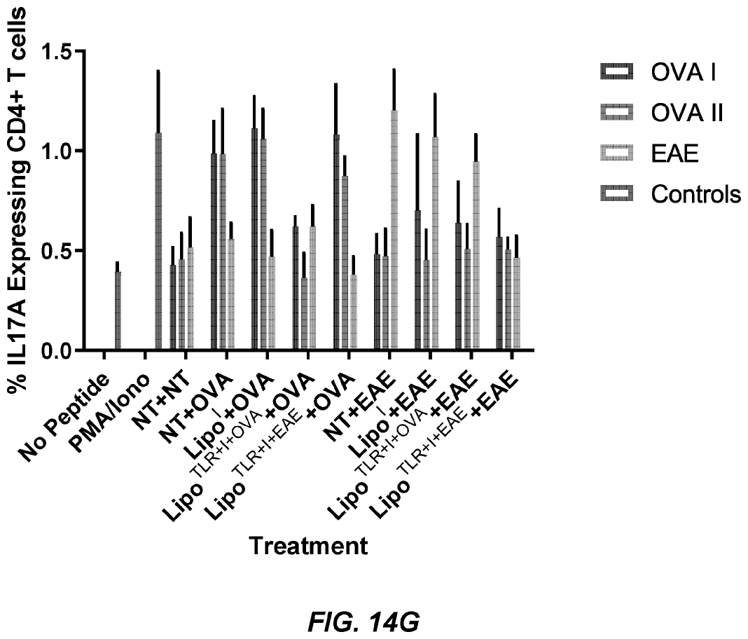

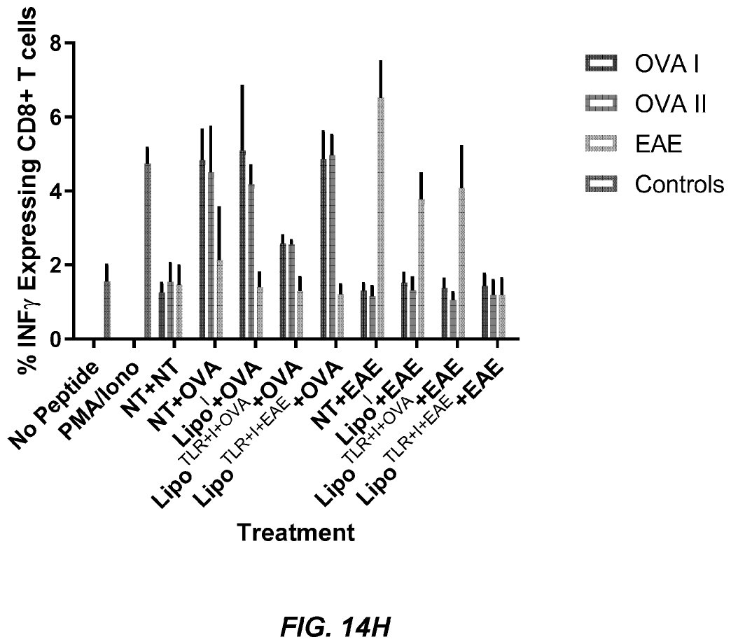

While this data strongly supported that the tolerogenic liposome formulation suppresses immunity and generates Tregs and tolDCs, the inventors sought to confirm that the suppression was selectively antigen specific. To show this selectivity, the inventors prepared two different antigen-bearing formulations of Lipo^{TLR+I}, one with OVA and one with the major peptide epitope of myelin oligodendrocyte glycoprotein (MOG₃₅₋₅₅). Then mice were challenged with either CpG/OVA or with the mouse model experimental autoimmune encephalomyelitis (EAE) that generate specific immune responses against MOG (see FIG. 3E schematic). It was hypothesized that mice tolerized against OVA would not prevent usual immune responses against EAE and vice versa, but would generate bystander tolerance against the antigen included in the treatment. After mice were sacrificed on day 15, blood was analyzed for MOG and OVA specific IgGs and inguinal lymph nodes analyzed for antigen specific Tregs using tetramers to the major MHCII epitopes of MOG and OVA (FIGS. 3E-3H) As expected, OVA-Lipo^{TLR+I} significantly increased OVA-specific Treg populations compared to CpG/OVA challenge alone, while MOG-Lipo^{TLR+I} did not (FIG. 3E). A similar trend was seen with anti-OVA IgG, with OVA specific treatments reducing OVA-IgGs but MOG specific treatments not (FIG. 3F). The antigen specificity was further confirmed when MOG-Lipo^{TLR}+I only increased MOG specific Tregs and decreased MOG IgGs (FIGS. 3G and 3H, see FIGS. 14A-14H for further analysis). Antigen specificity was also seen in intracellular staining (ICS) of splenocytes from these mice, although it should be noted that while Lipo^{TLR+I} significantly reduced INFγ+ and IL17A+CD4 T cells and INFγ+CD8 T cells in an antigen specific fashion, liposomal treatments had less of an effect on IL4+CD4+ T cells (FIGS. 14A-14H). Overall, these data provided strong evidence that Lipo^{TLR+I} generated antigen specific bystander tolerance.

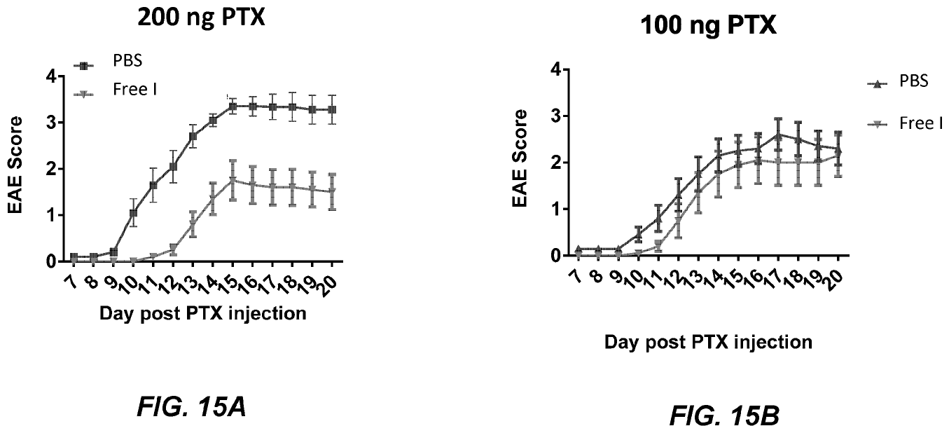

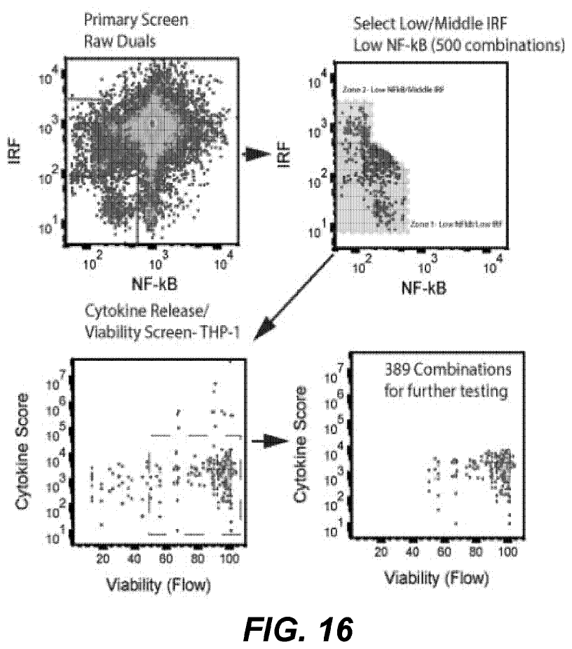

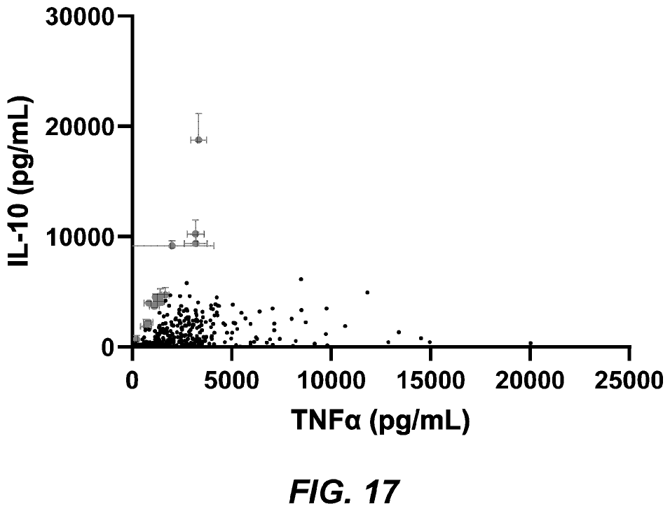

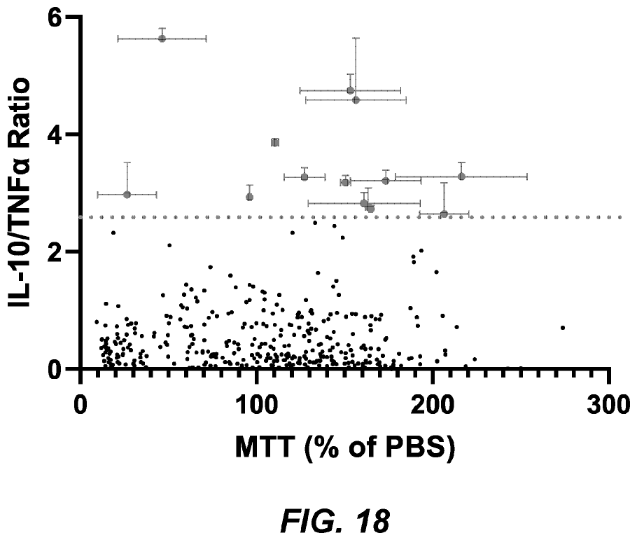

The last goal of these studies was to validate the tolerance system in a mouse model of an autoimmune disease. The mouse model experimental autoimmune encephalomyelitis (EAE) was chosen—a model of multiple sclerosis (MS)(28). In this model, a small peptide specific to myelin oligodendrocyte glycoprotein (MOG) in mice is injected with a combination of strong immune stimulants to generate antigen specific T and IgG responses that destroy mouse oligodendrocytes—ultimately resulting in severe paralysis. First, a small pilot study was performed where groups of 10 mice were given a prophylactic treatment of liposomes with tolerance compounds then induced to generate EAE autoimmune responses and compared to no pretreatment. This study determined the optimal method for inducing EAE and that the inhibitor combination can reduce EAE responses in a pretreatment (FIGS. 15A and 15B).

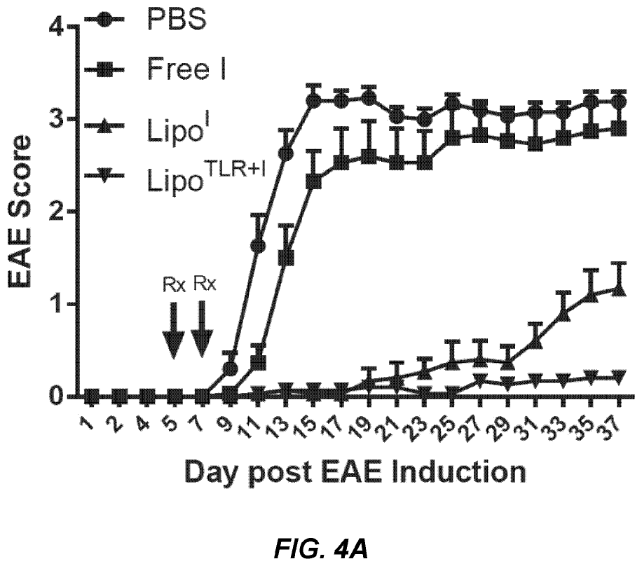

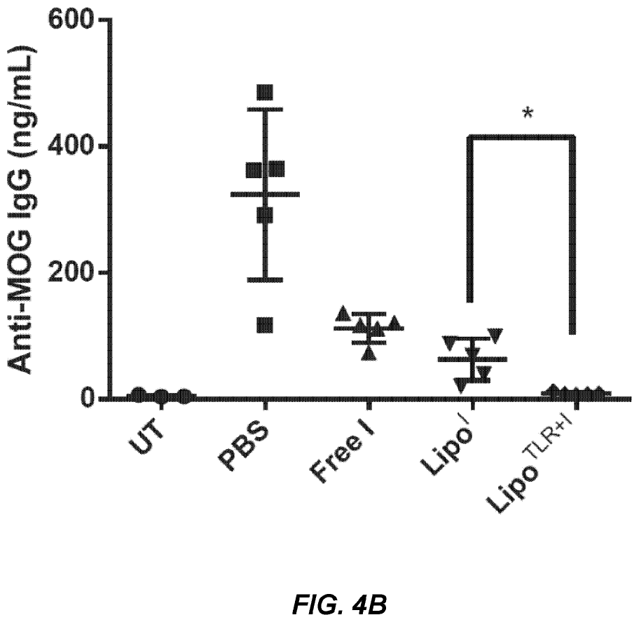

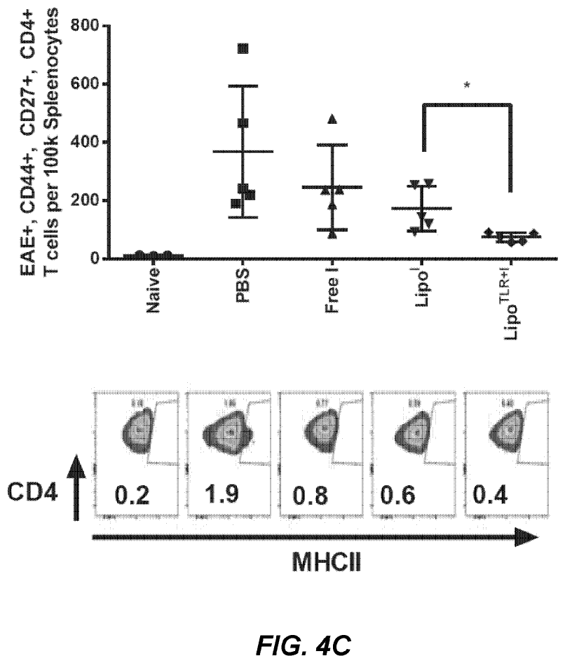

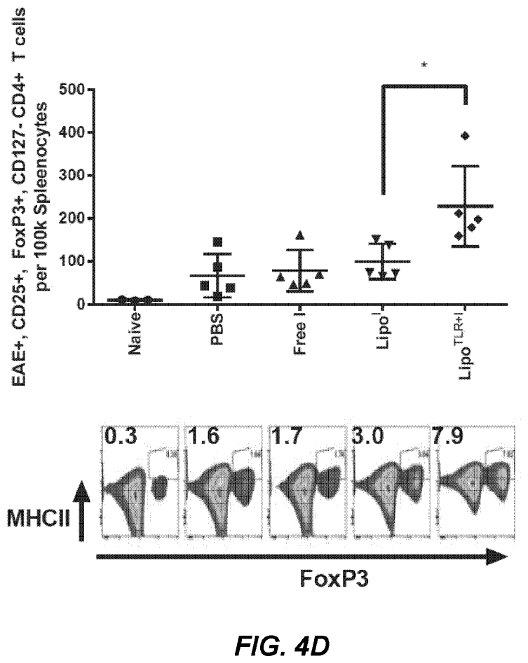

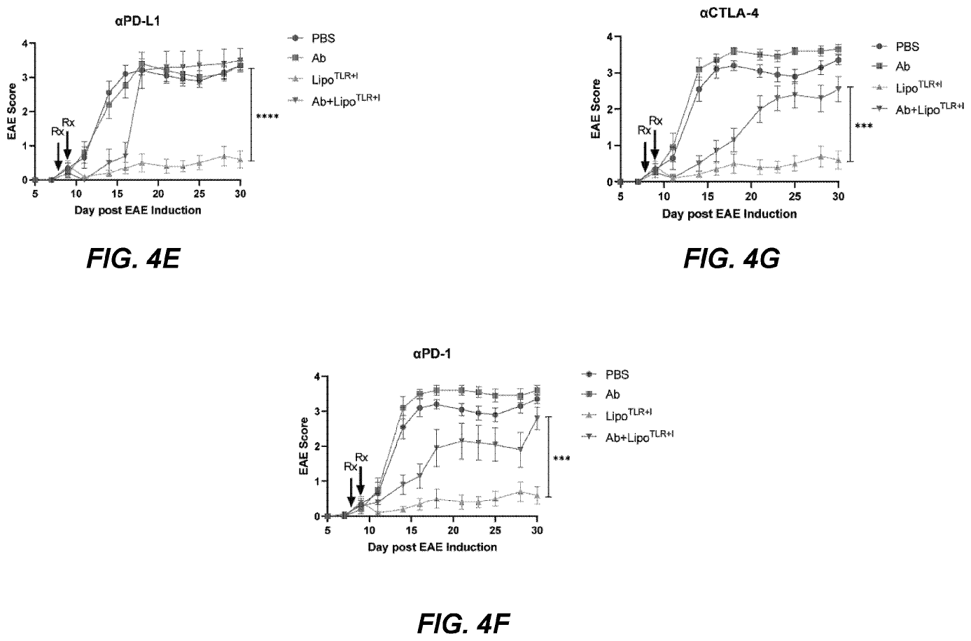

A more clinically valuable experiment is induced EAE responses that are then treated; as most autoimmune diseases are not diagnosed until after symptoms arise. The inventors took mice (n=14-15 per group) and induced EAE, then injected with either the MOG₃₅₋₅₅+Lipo^{TLR+I}, MOG₃₅₋₅₅+ Lipo^{I}, MOG₃₅₋₅₅+ free I, or PBS mock injections. Mice were injected with the two dosing regimen similar to that of the studies shown in FIG. 3 on day 4/5 and the treatment repeated on day 6/7, then EAE symptoms were monitored for 30 days after final injection (FIG. 4A). Lipo^{TLR+I} and Lipo^{l} formulations prevented strong EAE symptoms from appearing almost entirely, but after three weeks the Lipo^{I} formulation began to show slight symptoms while the Lipo^{TLR+I} formulation showed no significant symptoms. This is important to note, as the push/pull system of TLR+I provided longer term symptom protection than just I alone which seemed to deteriorate after 3 weeks. Groups of 3-5 mice were similarly treated but sacrificed on day 14 after EAE induction to observe anti-MOG IgG titers, CD4⁺ r EAE tetramerr T cell and MOG₃₅₋₅₅ Tetramer+ Treg populations. The Lipo^{TLR+I} group showed the lowest anti-MOG IgG, lowest CD4⁺ MOG₃₅₋₅₅ tetramer⁺ T cells and highest EAE⁺ Treg populations, demonstrating that the formulation was protecting mice from symptoms through a Treg mechanism (FIGS. 4B-4D). Finally, splenocytes from these groups had the smallest levels of T cell proliferation in response to antigen challenge and lowest levels of inflammatory/T cell cytokines (FIGS. 4E and 4F). All of this data provides strong evidence that this Lipo^{TLR+I} formulation can generate strong antigen specific T regs, which can abrogate autoimmune responses in a relevant disease model.

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. J. M. Carballido, C. Regairaz, C. Rauld, L. Raad, D. Picard, M. Kammüller, The Emerging Jamboree of Transformative Therapies for Autoimmune Diseases. Front Immunol. 11 (2020), doi:10.3389/fimmu.2020.00472.
2. S. M. Hayter, M. C. Cook, Updated assessment of the prevalence, spectrum and case definition of autoimmune disease. Autoimmun Rev. 11, 754-765 (2012).
3. P. Serra, P. Santamaria, Antigen-specific therapeutic approaches for autoimmunity. Nature Biotechnology. 37, 238-251 (2019).
4. M. Romano, G. Fanelli, C. J. Albany, G. Giganti, G. Lombardi, Past, Present, and Future of Regulatory T Cell Therapy in Transplantation and Autoimmunity. Front. Immunol. 10 (2019), doi:10.3389/fimmu.2019.00043.
5. B. O. Roep, N. Solvason, P. A. Gottlieb, J. R. F. Abreu, L. C. Harrison, G. S. Eisenbarth, L. Yu, M. Leviten, W. A. Hagopian, J. B. Buse, M. von Herrath, J. Quan, R. S. King, W. H. Robinson, P. J. Utz, H. Garren, BHT-3021 Investigators, L. Steinman, Plasmid-encoded proinsulin preserves C-peptide while specifically reducing proinsulin-specific CD8+ T cells in type 1 diabetes. Sci Transl Med. 5, 191ra82 (2013).
6. J. Chen, Q. Wang, D. Yin, V. Vu, R. Sciammas, A. S. Chong, Cutting Edge: CTLA-4Ig Inhibits Memory B Cell Responses and Promotes Allograft Survival in Sensitized Recipients. The Journal of Immunology. 195, 4069-4073 (2015).
7. M. P. Domogalla, P. V. Rostan, V. K. Raker, K. Steinbrink, Tolerance through Education: How Tolerogenic Dendritic Cells Shape Immunity. Front. Immunol. 8 (2017), doi:10.3389/fimmu.2017.01764.
8. S. Gregori, D. Tomasoni, V. Pacciani, M. Scirpoli, M. Battaglia, C. F. Magnani, E. Hauben, M.-G. Roncarolo, Differentiation of type 1 T regulatory cells (Tr1) by tolerogenic DC-10 requires the IL-10-dependent ILT4/HLA-G pathway. Blood. 116, 935-944 (2010).
9. J. J. Cho, J. M. Stewart, T. T. Drashansky, M. A. Brusko, A. N. Zuniga, K. J. Lorentsen, B. G. Keselowsky, D. Avram, An antigen-specific semi-therapeutic treatment with local delivery of tolerogenic factors through a dual-sized microparticle system blocks experimental autoimmune encephalomyelitis. Biomaterials. 143, 79-92 (2017).
10. J. S. Lewis, J. M. Stewart, G. P. Marshall, M. R. Carstens, Y. Zhang, N. V. Dolgova, C. Xia, T. M. Brusko, C. H. Wasserfall, M. J. Clare-Salzler, M. A. Atkinson, B. G. Keselowsky, Dual-Sized Microparticle System for Generating Suppressive Dendritic Cells Prevents and Reverses Type 1 Diabetes in the Nonobese Diabetic Mouse Model. ACS Biomater. Sci. Eng. 5, 2631-2646 (2019).
11. D. Odobasic, V. Oudin, K. Ito, P.-Y. Gan, A. R. Kitching, S. R. Holdsworth, Tolerogenic Dendritic Cells Attenuate Experimental Autoimmune Antimyeloperoxidase Glomerulonephritis. J Am Soc Nephrol. 30, 2140-2157 (2019).
12. M. A. Boks, J. R. Kager-Groenland, M. S. P. Haasjes, J. J. Zwaginga, S. M. van Ham, A. ten Brinke, IL-10-generated tolerogenic dendritic cells are optimal for functional regulatory T cell induction—A comparative study of human clinical-applicable DC. Clinical Immunology. 142, 332-342 (2012).
13. B. Pulendran, H. Tang, S. Manicassamy, Programming dendritic cells to induce T(H)2 and tolerogenic responses. Nat. Immunol. 11, 647-655 (2010).
14. C. Obregon, R. Kumar, M. A. Pascual, G. Vassalli, D. Golshayan, Update on Dendritic Cell-Induced Immunological and Clinical Tolerance. Front Immunol. 8 (2017), doi:10.3389/fimmu.2017.01514.
15. Y. Xing, K. A. Hogquist, T-Cell Tolerance: Central and Peripheral. Cold Spring Harb Perspect Biol. 4, a006957 (2012).
16. S. Akira, H. Hemmi, Recognition of pathogen-associated molecular patterns by TLR family. Immunology Letters. 85, 85-95 (2003).
17. A. F. de Vos, J. M. Pater, P. S. van den Pangaart, M. D. de Kruif, C. van't Veer, T. van der Poll, In vivo lipopolysaccharide exposure of human blood leukocytes induces cross-tolerance to multiple TLR ligands. J. Immunol. 183, 533-542 (2009).
18. Frontiers|Generation and Function of Induced Regulatory T Cells|Immunology, (available on the world wide web at frontiersin.org/articles/10.3389/fimmu.2013.00152/full).
19. S. Akira, K. Takeda, Toll-like receptor signalling. Nat Rev Immunol. 4, 499-511 (2004).
20. A. M. Dudek, S. Martin, A. D. Garg, P. Agostinis, Immature, Semi-Mature, and Fully Mature Dendritic Cells: Toward a DC-Cancer Cells Interface That Augments Anticancer Immunity. Front Immunol. 4 (2013), doi:10.3389/fimmu.2013.00438.
21. Y. You, Y. Qin, X. Lin, F. Yang, J. Li, S. R. Sooranna, L. Pinhu, Methylprednisolone attenuates lipopolysaccharide-induced Fractalkine expression in kidney of Lupus-prone MRL/lpr mice through the NF-kappaB pathway. BMC Nephrol. 16 (2015), doi:10.1186/s12882-015-0145-y.
22. T. Plesner, H.-T. Arkenau, P. Gimsing, J. Krejcik, C. Lemech, M. C. Minnema, U. Lassen, J. P. Laubach, A. Palumbo, S. Lisby, L. Basse, J. Wang, A. K. Sasser, M. E. Guckert, C. de Boer, N. Z. Khokhar, H. Yeh, P. L. Clemens, T. Ahmadi, H. M. Lokhorst, P. G. Richardson, Phase 1/2 study of daratumumab, lenalidomide, and dexamethasone for relapsed multiple myeloma. Blood. 128, 1821-1828 (2016).
23. T. Aprahamian, R. Bonegio, J. Rizzo, H. Perlman, D. J. Lefer, I. R. Rifkin, K. Walsh, Simvastatin Treatment Ameliorates Autoimmune Disease Associated with Accelerated Atherosclerosis in a Murine Lupus Model. J Immunol. 177, 3028-3034 (2006).
24. T. K. Kishimoto, R. A. Maldonado, Nanoparticles for the Induction of Antigen-Specific Immunological Tolerance. Front Immunol. 9 (2018), doi:10.3389/fimmu.2018.00230.
25. R. Galea, H. J. Nel, M. Talekar, X. Liu, J. D. Ooi, M. Huynh, S. Hadjigol, K. J. Robson, Y. T. Ting, S. Cole, K. Cochlin, S. Hitchcock, B. Zeng, S. Yekollu, M. Boks, N. Goh, H. Roberts, J. Rossjohn, H. H. Reid, B. J. Boyd, R. Malaviya, D. J. Shealy, D. G. Baker, L. Madakamutil, A. R. Kitching, B. J. O'Sullivan, R. Thomas, PD-L1- and calcitriol-dependent liposomal antigen-specific regulation of systemic inflammatory autoimmune disease. JCI Insight. 4 (2019), doi:10.1172/jci.insight.126025.
26. C. D. Andrews, C. J. Provoda, G. Ott, K.-D. Lee, Conjugation of lipid and CpG-containing oligonucleotide yields an efficient method for liposome incorporation. Bioconjug Chem. 22, 1279-1286 (2011).
27. M. Bartneck, F. M. Peters, K. T. Warzecha, M. Bienert, L. van Bloois, C. Trautwein, T. Lammers, F. Tacke, Liposomal encapsulation of dexamethasone modulates cytotoxicity, inflammatory cytokine response, and migratory properties of primary human macrophages. Nanomedicine: Nanotechnology, Biology and Medicine. 10, 1209-1220 (2014).
28. C. S. Constantinescu, N. Faroogi, K. O'Brien, B. Gran, Experimental autoimmune encephalomyelitis (EAE) as a model for multiple sclerosis (MS). Br J Pharmacol. 164, 1079-1106 (2011).
29. L. M. Nelson, M. T. Wallin, R. A. Marrie, W. J. Culpepper, A. Langer-Gould, J. Campbell, S. Buka, H. Tremlett, G. Cutter, W. Kaye, L. Wagner, N. G. Larocca, for the U. S. M. S. P. Workgroup, A new way to estimate neurologic disease prevalence in the United States: Illustrated with MS. Neurology. 92, 469-480 (2019).
30. C. Krienke, L. Kolb, E. Diken, M. Streuber, S. Kirchhoff, T. Bukur, Ö. Akilli-Öztürk, L. M. Kranz, H. Berger, J. Petschenka, M. Diken, S. Kreiter, N. Yogev, A. Waisman, K. Karikó, Ö. Türeci, U. Sahin, A noninflammatory mRNA vaccine for treatment of experimental autoimmune encephalomyelitis. Science. 371, 145-153 (2021).
31. Y. M. Ahmed, B. A. S. Messiha, A. A. Abo-Saif, Protective Effects of Simvastatin and Hesperidin against Complete Freund's Adjuvant-Induced Rheumatoid Arthritis in Rats. Pharmacology. 96, 217-225 (2015).
32. K. Maneechotesuwan, K. Kasetsinsombat, V. Wamanuttajinda, A. Wongkajornsilp, P. J. Barnes, Statins enhance the effects of corticosteroids on the balance between regulatory T cells and Th17 cells. Clin Exp Allergy. 43, 212-222 (2013).
33. K. Maneechotesuwan, W. Ekjiratrakul, K. Kasetsinsombat, A. Wongkajornsilp, P. J. Barnes, Statins enhance the anti-inflammatory effects of inhaled corticosteroids in asthmatic patients through increased induction of indoleamine 2, 3-dioxygenase. J Allergy Clin Immunol. 126, 754-762.e1 (2010).
34. S. Chamorro, J. J. Garcia-Vallejo, W. W. J. Unger, R. J. Fernandes, S. C. M. Bruijns, S. Laban, B. O. Roep, B. A. 't Hart, Y. van Kooyk, TLR Triggering on Tolerogenic Dendritic Cells Results in TLR2 Up-Regulation and a Reduced Proinflammatory Immune Program. The Journal of Immunology. 183, 2984-2994 (2009).
35. Y.-L. Xue, S.-X. Zhang, C.-F. Zheng, Y.-F. Li, L.-H. Zhang, Y.-F. Hao, S. Wang, X.-W. Li, Silencing of STAT4 Protects Against Autoimmune Myocarditis by Regulating Th1/Th2 Immune Response via Inactivation of the NF-κB Pathway in Rats. Inflammation. 42, 1179-1189 (2019).
36. S. Martini, M. Nielsen, B. Peters, A. Sette, The Immune Epitope Database and Analysis Resource Program 2003-2018: reflections and outlook. Immunogenetics. 72, 57-76 (2020).

1. A composition comprising (a) a TLR agonist; and (b) one or more immunosuppressive agents.

2. The composition of claim 1, wherein the composition further comprises an antigen.

3. The composition of claim 1 or 2, wherein the one or more immunosuppressive agents comprise one or more of cucurbitacin I, costunolide, MLN120B, parthenolide, peficitinib, oclacitinib maleate, AD80, cucurbitacin B, CEP-33779, Dehydrocostus Lactone, dexamethasone, simvastatin, SC 514, ly294002, minocycline, hydroxychloroquine, Sialyl Lewis X, Thymic Stromal Lymphopoietin (TSLP), and lifitegrast.

4. The composition of claim 3, wherein the one or more immunosuppressive agents comprise one or more of dexamethasone, simvastatin, and SC 514.

5. The composition of claim 4, wherein the one or more immunosuppressive agents comprise two or more of dexamethasone, simvastatin, and SC 514.

6. The composition of claim 5, wherein the one or more immunosuppressive agents comprise dexamethasone, simvastatin, and SC 514.

7. The composition of claim 6, wherein the dexamethasone, simvastatin, and SC 514 are at a 1:1:1 concentration ratio.

8. The composition of any of claims 1-7, wherein the TLR agonist is a TLR5 agonist, a TLR9 agonist, a TLR2 agonist, a TLR6 agonist, a TLR2/6 agonist, a TLR7 agonist, a TLR8 agonist, a TLR 7/8 agonist, or a TLR4 agonist.

9. The composition of claim 8, wherein the TLR agonist comprises a TLR5 agonist.

10. The composition of claim 9, wherein the TLR agonist comprises flagellin.

11. The composition of claim 10, wherein the flagellin comprises Bacillus subtilis flagellin.

12. The composition of claim 8, wherein the TLR agonist comprises a TLR9 agonist.

13. The composition of claim 12, wherein the TLR agonist comprises a CpG oligonucleotide (CpG ODN).

14. The composition of claim 13, wherein the CpG oligonucleotide comprises CpG ODN 1826.

15. The composition of any one of claims 1-14, wherein the TLR agonist comprises LPS.

16. The composition of any one of claims 1-15, wherein the TLR agonist comprises Pam2CSK4.

17. The composition of any one of claims 1-16, wherein the TLR agonist comprises R848.

18. The composition of any one of claims 1-17, wherein the composition comprises cucurbitacin I and LPS.

19. The composition of any one of claims 1-18, wherein the composition comprises costunolide and Pam2CSK4.

20. The composition of any one of claims 1-19, wherein the composition comprises MLN120B and Pam2CSK4.

21. The composition of any one of claims 1-20, wherein the composition comprises parthenolide and Pam2CSK4.

22. The composition of any one of claims 1-21, wherein the composition comprises peficitinib and Pam2CSK4.

23. The composition of any one of claims 1-22, wherein the composition comprises oclacitinib maleate and R848.

24. The composition of any one of claims 1-23, wherein the composition comprises AD80 and Pam2CSK4.

25. The composition of any one of claims 1-24, wherein the composition comprises curcurbitacin B and R848.

26. The composition of any one of claims 1-25, wherein the composition comprises CEP-33779 and R848.

27. The composition of any one of claims 1-26, wherein the composition comprises dehydrocostus lactone and R848.

28. The composition of any of claims 2-27, wherein the antigen is a cancer antigen.

29. The composition of any of claims 2-27, wherein the antigen is an antigen associated with an autoimmune condition.

30. The composition of claim 29, wherein the autoimmune condition is multiple sclerosis.

31. The composition of claim 29 or 30, wherein the antigen is a myelin sheath protein or portion thereof.

32. The composition of claim 31, wherein the myelin sheath protein is myelin basic protein (MBP), myelin oligodendrocyte glycoprotein (MOG), proteolipid protein (PLP), or myelin associated glycoprotein (MAG).

33. The composition of claim 32, wherein the antigen is a MOG peptide.

34. The composition of any one of claims 1-33, wherein the molar ratio of TLR agonist to immunosuppressive agent is from 1:10-1:100.

35. The composition of any one of claims 1-33, wherein the molar ratio of TLR agonist to immunosuppressive agent is from 5:1-20:1.

36. The composition of any one of claims 1-35, wherein the concentration of the immunosuppressive agent is 0.1 μM-10 μM.

37. The composition of any one of claims 1-35, wherein the concentration of the TLR agonist(s) is 1 nM-1 μM.

38. A nanocarrier comprising the composition of any one of claims 1-37.

39. The nanocarrier of claim 38, wherein the nanocarrier is a liposome, a nanoparticle, a dendrimer, or a micelle.

40. The nanocarrier of claim 39, wherein the nanocarrier is a liposome.

41. The nanocarrier of any one of claims 38-40, wherein the nanocarrier is a liposome, wherein the TLR agonist is conjugated to the liposome.

42. The nanocarrier of any one of claims 38-41, wherein the nanocarrier is a liposome, wherein the TLR agonist is displayed on a surface of the liposome.

43. The nanocarrier of any of claims 38-42, wherein the nanocarrier is a liposome, wherein the antigen is encapsulated in an interior of the liposome.

44. The nanocarrier of any of claims 38-43, wherein the nanocarrier is a liposome, wherein the one or more immunosuppressive agents are encapsulated in a membrane of the liposome.

45. A pharmaceutical composition comprising (i) the composition of any one of claims 1-37 or the nanocarrier of any of claims 38-44 and (ii) a pharmaceutically acceptable excipient.

46. A method for treating a subject for cancer or an autoimmune or inflammatory condition, the method comprising administering to the subject an effective amount of the composition of any one of claims 1-37, the nanocarrier of any of claims 38-44, or the pharmaceutical composition of claim 45.

47. A method for treating a subject for cancer or an autoimmune or inflammatory condition, the method comprising administering to the subject an effective amount of
(i) one or more TLR agonist; and
(ii) one or more immunosuppressive agents.

48. The method of claim 47, wherein the method further comprises administering an antigen.

49. The method of claim 47, wherein the one or more immunosuppressive agents comprise one or more of cucurbitacin I, costunolide, MLN120B, parthenolide, peficitinib, oclacitinib maleate, AD80, cucurbitacin B, CEP-33779, Dehydrocostus Lactone, dexamethasone, simvastatin, SC 514, ly294002, minocycline, hydroxychloroquine, Sialyl Lewis X, Thymic Stromal Lymphopoietin (TSLP), and lifitegrast.

50. The method of claim 49, wherein the one or more immunosuppressive agents comprise one or more of dexamethasone, simvastatin, and SC 514.

51. The method of claim 50, wherein the one or more immunosuppressive agents comprise two or more of dexamethasone, simvastatin, and SC 514.

52. The method of claim 51, wherein the one or more immunosuppressive agents comprise dexamethasone, simvastatin, and SC 514.

53. The method of claim 52, wherein the dexamethasone, simvastatin, and SC 514 are at a 1:1:1 concentration ratio.

54. The method of any one of claims 47-53, wherein the TLR agonist is a TLR5 agonist, a TLR9 agonist, a TLR2 agonist, a TLR6 agonist, a TLR2/6 agonist, a TLR7 agonist, a TLR8 agonist, a TLR 7/8 agonist, or a TLR4 agonist.

55. The method of claim 54, wherein the TLR agonist comprises a TLR5 agonist.

56. The method of claim 55, wherein the TLR agonist comprises flagellin.

57. The method of claim 56, wherein the flagellin comprises Bacillus subtilis flagellin.

58. The method of any one of claims 54-57, wherein the TLR agonist comprises a TLR9 agonist.

59. The method of claim 58, wherein the TLR agonist comprises a CpG oligonucleotide (CpG ODN).

60. The method of claim 59, wherein the CpG oligonucleotide comprises CpG ODN 1826.

61. The method of any one of claims 47-60, wherein the TLR agonist comprises LPS.

62. The method of any one of claims 47-61, wherein the TLR agonist comprises Pam2.

63. The method of any one of claims 47-62, wherein the TLR agonist comprises R848.

64. The method of any one of claims 47-63, wherein the subject is administered cucurbitacin I and LPS.

65. The method of any one of claims 47-64, wherein the subject is administered costunolide and Pam2CSK4.

66. The method of any one of claims 47-65, wherein the subject is administered MLN120B and Pam2CSK4.

67. The method of any one of claims 47-66, wherein the subject is administered parthenolide and Pam2CSK4.

68. The method of any one of claims 47-67, wherein the subject is administered peficitinib and Pam2CSK4.

69. The method of any one of claims 47-68, wherein the subject is administered oclacitinib maleate and R848.

70. The method of any one of claims 47-69, wherein the subject is administered AD80 and Pam2CSK4.

71. The method of any one of claims 47-70, wherein the subject is administered curcurbitacin B and R848.

72. The method of any one of claims 47-71, wherein the subject is administered CEP-33779 and R848.

73. The method of any one of claims 47-72, wherein the subject is administered dehydrocostus lactone and R848.

74. The method of any of claims 48-73, wherein the antigen is a cancer antigen.

75. The method of any of claims 48-73, wherein the antigen is an antigen associated with an autoimmune condition.

76. The method of claim 75, wherein the autoimmune condition is multiple sclerosis.

77. The method of claim 75 or 76, wherein the antigen is a myelin sheath protein or portion thereof.

78. The method of claim 77, wherein the myelin sheath protein is myelin basic protein (MBP), myelin oligodendrocyte glycoprotein (MOG), proteolipid protein (PLP), or myelin associated glycoprotein (MAG).

79. The method of claim 78, wherein the antigen is a MOG peptide.

80. The method of any of claims 48-79, wherein the antigen is a self-antigen.

81. The method of any of claims 48-79, wherein the antigen is a foreign antigen.

82. The method of any one of claims 47-81, wherein the molar ratio of administered TLR agonist to administered immunosuppressive agent is from 1:10-1:100.

83. The method of any one of claims 47-81, wherein the molar ratio of administered TLR agonist to administered immunosuppressive agent is from 5:1-20:1.

84. The method of any one of claims 47-83, wherein the TLR agonist(s), immunosuppressive agent(s) and optionally antigen are administered in a nanocarrier.

85. The method of any one of claims 47-84, wherein the TLR agonist(s), immunosuppressive agent(s) and/or nanocarrier is administered to the subject prior to, together with, or after an allogeneic transplant.

86. The method of claim 85, wherein the method is for preventing or treating graft versus host disease or graft rejection.

87. The method of claim 85 or 86, wherein the subject is administered an antigen, and wherein the antigen is an antigen from a graft of the transplant.

88. The method of claim 85 or 87, wherein the allogeneic transplant is a bone marrow transplant.

89. The method of claim 88, wherein the subject is administered a bone marrow antigen.

90. The method of any of claims 85-89, wherein the allogeneic transplant is an organ transplant.

91. The method of claim 90, wherein the subject is administered an antigen from the organ.

92. The method of any of claims 47-91, wherein the method further comprising administering to the subject an effective amount of
(1) a second TLR agonist; and
(2) a second immunosuppressive agent.

93. The method of claim 92, wherein the method further comprises administering a second antigen associated with the autoimmune or inflammatory condition.

94. The method of claim 92 or 93, wherein the second immunosuppressive agent comprises one or more of cucurbitacin I, costunolide, MLN120B, parthenolide, peficitinib, oclacitinib maleate, AD80, cucurbitacin B, CEP-33779, Dehydrocostus Lactone, dexamethasone, simvastatin, SC 514, ly294002, minocycline, hydroxychloroquine, Sialyl Lewis X, Thymic Stromal Lymphopoietin (TSLP), and lifitegrast.

95. The method of claim 93, wherein the one or more immunosuppressive agents comprise one or more of dexamethasone, simvastatin, and SC 514.

96. The method of claim 95, wherein the one or more immunosuppressive agents comprise two or more of dexamethasone, simvastatin, and SC 514.

97. The method of claim 96, wherein the one or more immunosuppressive agents comprise dexamethasone, simvastatin, and SC 514.

98. The method of claim 97, wherein the dexamethasone, simvastatin, and SC 514 are at a 1:1:1 concentration ratio.

99. The method of any one of claims 92-98, wherein the second TLR agonist is a TLR5 agonist, a TLR9 agonist, a TLR2 agonist, a TLR6 agonist, a TLR2/6 agonist, a TLR7 agonist, a TLR8 agonist, a TLR 7/8 agonist, or a TLR4 agonist.

100. The method of claim 99, wherein the second TLR agonist comprises a TLR5 agonist.

101. The method of claim 100, wherein the second TLR agonist comprises flagellin.

102. The method of claim 101, wherein the flagellin comprises Bacillus subtilis flagellin.

103. The method of any one of claims 92-102, wherein the second TLR agonist comprises a TLR9 agonist.

104. The method of claim 103, wherein the second TLR agonist comprises a CpG oligonucleotide (CpG ODN).

105. The method of claim 104, wherein the CpG oligonucleotide comprises CpG ODN 1826.

106. The method of any one of claims 92-105, wherein the second TLR agonist comprises LPS.

107. The method of any one of claims 92-106, wherein the second TLR agonist comprises Pam2CSK4.

108. The method of any one of claims 92-107, wherein the second TLR agonist comprises R848.

109. The method of any one of claims 92-108, wherein the subject is administered cucurbitacin I and LPS.

110. The method of any one of claims 92-109, wherein the subject is administered costunolide and Pam2CSK4.

111. The method of any one of claims 92-110, wherein the subject is administered MLN120B and Pam2CSK4.

112. The method of any one of claims 92-111, wherein the subject is administered parthenolide and Pam2CSK4.

113. The method of any one of claims 92-112, wherein the subject is administered peficitinib and Pam2CSK4.

114. The method of any one of claims 92-113, wherein the subject is administered oclacitinib maleate and R848.

115. The method of any one of claims 92-114, wherein the subject is administered AD80 and Pam2CSK4.

116. The method of any one of claims 92-115, wherein the subject is administered curcurbitacin B and R848.

117. The method of any one of claims 92-116, wherein the subject is administered CEP-33779 and R848.

118. The method of any one of claims 92-105, wherein the subject is administered dehydrocostus lactone and R848.

119. The method of any of claims 92-118, wherein the second antigen is a cancer antigen or an antigen associated with an autoimmune condition.

120. The method of claim 119, wherein the autoimmune condition is multiple sclerosis.

121. The method of claim 119 or 120, wherein the second antigen is a myelin sheath protein or portion thereof.

122. The method of claim 121, wherein the myelin sheath protein is myelin basic protein (MBP), myelin oligodendrocyte glycoprotein (MOG), proteolipid protein (PLP), or myelin associated glycoprotein (MAG).

123. The method of claim 122, wherein the second antigen is a MOG peptide.

124. The method of any of claims 93-123, wherein the second antigen is a self-antigen.

125. The method of any of claims 93-123, wherein the second antigen is a foreign antigen.

126. The method of any one of claims 92-125, wherein the molar ratio of administered second TLR agonist to administered second immunosuppressive agent is from 1:10-1:100.

127. The method of any one of claims 92-125, wherein the molar ratio of administered second TLR agonist to administered second immunosuppressive agent is from 5:1-20:1.

128. The method of any one of claims 92-119, wherein the second immunosuppressive agent, second TLR agonist and optional second antigen is administered in a second nanocarrier composition.

129. The method of claim 128, wherein the second nanocarrier is a liposome, a nanoparticle, a dendrimer, or a micelle.

130. The method of any of claim 129, wherein the second nanocarrier is a liposome.

131. The method of any of claims 92-130, wherein the TLR agonist, immunosuppressive agent, and optional antigen are administered at the same time as the second TLR agent, second immunosuppressive agent, and optional second antigen.

132. The method of any of claims 92-130, wherein the TLR agonist, immunosuppressive agent, and optional antigen are administered before or after the second TLR agent, second immunosuppressive agent, and optional second antigen.

133. The method of claim 132, wherein the second TLR agent, second immunosuppressive agent, and optional second antigen is administered to the subject at least 12, 18, or 24 hours before or after administering the TLR agonist, immunosuppressive agent, and optional antigen to the subject.

134. The method of any of claims 128-133, wherein the second nanocarrier is a liposome, wherein the additional antigen is encapsulated in an interior of the liposome.

135. The method of any of claims 128-133, wherein the second nanocarrier is a liposome, wherein the one or more second immunosuppressive agents are encapsulated in a surface membrane of the liposome.

136. The method of any one of claims 46-135, wherein the method is for treating cancer.

137. The method of claim 136, wherein the method further comprises administering an additional cancer therapy.

138. The method of claim 137, wherein the additional cancer therapy comprises an immunotherapy.

139. A method for generating tolerogenic dendritic cells, the method comprising providing, to a population of dendritic cells or dendritic cell precursors:
(a) one or more TLR agonists; and
(b) one or more immunosuppressive agents.

140. The method of claim 139, wherein the population of dendritic cells or dendritic cell precursors are incubated with the composition for at least 12 hours.

141. The method of claim 139, wherein the population of dendritic cells or dendritic cell precursors are incubated with the composition for at least 18 hours.

142. The method of claim 139, wherein the population of dendritic cells or dendritic cell precursors are incubated with the composition for at least 24 hours.

143. The method of any of claims 139-142, wherein the one or more immunosuppressive agents comprise one or more of cucurbitacin I, costunolide, MLN120B, parthenolide, peficitinib, oclacitinib maleate, AD80, cucurbitacin B, CEP-33779, Dehydrocostus Lactone, dexamethasone, simvastatin, SC 514, ly294002, minocycline, hydroxychloroquine, Sialyl Lewis X, Thymic Stromal Lymphopoietin (TSLP), and lifitegrast.

144. The method of claim 143, wherein the one or more immunosuppressive agents comprise one or more of dexamethasone, simvastatin, and SC 514.

145. The method of claim 144, wherein the one or more immunosuppressive agents comprise two or more of dexamethasone, simvastatin, and SC 514.

146. The method of claim 145, wherein the one or more immunosuppressive agents comprise dexamethasone, simvastatin, and SC 514.

147. The method of claim 146, wherein the dexamethasone, simvastatin, and SC 514 are at a 1:1:1 concentration ratio.

148. The method of any one of claims 139-147, wherein the TLR agonist is a TLR5 agonist, a TLR9 agonist, a TLR2 agonist, a TLR6 agonist, a TLR2/6 agonist, a TLR7 agonist, a TLR8 agonist, a TLR 7/8 agonist, or a TLR4 agonist.

149. The method of claim 148, wherein the TLR agonist comprises a TLR5 agonist.

150. The method of claim 149, wherein the TLR agonist comprises flagellin.

151. The method of claim 150, wherein the flagellin comprises Bacillus subtilis flagellin.

152. The method of any one of claims 148-151, wherein the TLR agonist comprises a TLR9 agonist.

153. The method of claim 152, wherein the TLR agonist comprises a CpG oligonucleotide (CpG ODN).

154. The method of claim 153, wherein the CpG oligonucleotide comprises CpG ODN 1826.

155. The method of any one of claims 139-154, wherein the TLR agonist comprises LPS.

156. The method of any one of claims 139-155, wherein the TLR agonist comprises Pam2.

157. The method of any one of claims 139-156, wherein the TLR agonist comprises R848.

158. The method of any one of claims 139-157, wherein the cucurbitacin I and LPS is provided.

159. The method of any one of claims 139-158, wherein costunolide and Pam2CSK4 is provided.

160. The method of any one of claims 139-159, wherein MLN120B and Pam2CSK4 is provided.

161. The method of any one of claims 139-160, wherein parthenolide and Pam2CSK4 is provided.

162. The method of any one of claims 139-161, wherein peficitinib and Pam2CSK4 is provided.

163. The method of any one of claims 139-162, wherein oclacitinib maleate and R848 is provided.

164. The method of any one of claims 139-163, wherein AD80 and Pam2CSK4 is provided.

165. The method of any one of claims 139-164, wherein curcurbitacin B and R848 is provided.

166. The method of any one of claims 139-165, wherein CEP-33779 and R848 is provided.

167. The method of any one of claims 139-166, wherein dehydrocostus lactone and R848 is provided.

168. The method of any of claims 139-167, wherein the method comprises or further comprises providing an antigen to the population of dendritic cells or dendritic cell precursors.

169. The method of claim 168, wherein the antigen is a cancer antigen.

170. The method of claim 168, wherein the antigen is an antigen associated with an autoimmune condition.

171. The method of claim 170, wherein the autoimmune or inflammatory condition is multiple sclerosis.

172. The method of claim 171, wherein the antigen is a myelin sheath protein or portion thereof.

173. The method of claim 172, wherein the myelin sheath protein is myelin basic protein (MBP), myelin oligodendrocyte glycoprotein (MOG), proteolipid protein (PLP), or myelin associated glycoprotein (MAG).

174. The method of claim 173, wherein the antigen is a MOG peptide.

175. The method of any of claims 139-174, wherein the one or more TLR agonists and the one or more immunosuppressive agents are comprised in a nanocarrier.

176. The method of claim 175, wherein the nanocarrier is a liposome, a nanoparticle, a dendrimer, or a micelle.

177. The method of claim 176, wherein the nanocarrier is a liposome.

178. The method of claim 177, wherein the TLR agonist is conjugated to the liposome.

179. The method of claim 177 or 178, wherein the TLR agonist is displayed on a surface of the liposome.

180. The method of any of claims 177-179, wherein the nanocarrier further comprises an antigen, and wherein the antigen is encapsulated in an interior of the liposome.

181. The method of any of claims 177-179, wherein the nanocarrier further comprises an antigen, and the one or more immunosuppressive agents are encapsulated in a surface membrane of the liposome.

182. The method of any of claims 139-181, further comprising providing to the population of dendritic cells or dendritic cell precursors an additional composition comprising:
(a) one or more additional TLR agonists; and
(b) one or more additional immunosuppressive agents.

183. The method of claim 182, wherein the one or more TLR agonists comprise a TLR5 agonist and the one or more additional TLR agonists comprise a TLR9 agonist.

184. The method of claim 183, wherein the TLR5 agonist is flagellin.

185. The method of claim 183, wherein the TLT9 agonist is a CpG oligonucleotide.

186. The method of any of claims 182-185, wherein the additional composition is provided after administering the composition.

187. The method of claim 186, wherein the additional composition is provided at least 12 hours after administering the composition.

188. The method of claim 186, wherein the additional composition is provided at least 18 hours after administering the composition.

189. The method of claim 186, wherein the additional composition is provided at least 24 hours after administering the composition.

190. The method of any of claims 139-189, wherein the method is performed ex vivo.

191. The method of any of claims 139-189, wherein the method is performed in vivo.

192. The method of claim 191, wherein the composition is administered to the subject prior to, together with, or after an allogeneic transplant.

193. The method of claim 192, wherein the allogeneic transplant is a bone marrow transplant.

194. The method of any of claim 193, wherein the allogeneic transplant is an organ transplant.

195. The method of any one of claims 46-194, wherein the subject is a human subject.

196. A liposome comprising (a) an antigen associated with multiple sclerosis; (b) flagellin conjugated to an exterior surface of the liposome; (c) dexamethasone, (d) simvastatin, and (e) SC 514.

197. A liposome comprising (a) an antigen associated with multiple sclerosis; (b) a CpG oligonucleotide conjugated to an exterior surface of the liposome; (c) dexamethasone, (d) simvastatin, and (e) SC 514.

198. A method for treating a subject for multiple sclerosis, the method comprising administering to the subject an effective amount of a composition comprising:
a liposome comprising:
(i) an antigen associated with multiple sclerosis; and
(ii) the combination of one of:
(a) flagellin, dexamethasone, simvastatin, and SC 514;
(b) a CpG oligonucleotide, dexamethasone, simvastatin, and SC 514;
(c) cucurbitacin and LPS;
(d) costunolide and Pam2CSK4;
(e) MLN120B and Pam2CSK4;
(f) parthenolide and Pam2CSK4;
(g) peficitinib and Pam2CSK4;
(h) oclacitinib maleate and R848;
(i) AD80 and Pam2CSK4;
(j) cucurbitacin B and R848;
(k) CEP-33779 and R848; or
(l) dehydrocostus lactone and R848.

199. A method for treating or preventing graft versus host disease or graft rejection in a subject, the method comprising administering to the subject an effective amount of a composition comprising:
a liposome comprising the combination of one of:
(a) flagellin, dexamethasone, simvastatin, and SC 514;
(b) a CpG oligonucleotide, dexamethasone, simvastatin, and SC 514;
(c) cucurbitacin and LPS;
(d) costunolide and Pam2CSK4;
(e) MLN120B and Pam2CSK4;
(f) parthenolide and Pam2CSK4;
(g) peficitinib and Pam2CSK4;
(h) oclacitinib maleate and R848;
(i) AD80 and Pam2CSK4;
(j) cucurbitacin B and R848;
(k) CEP-33779 and R848; or
(l) dehydrocostus lactone and R848.

200. The method of claim 199, wherein the composition is administered to the subject prior to, together with, or after an allogeneic transplant.

201. The method of claim 200, wherein the allogeneic transplant is a bone marrow transplant.

202. The method of claim 200, wherein the allogeneic transplant is an organ transplant.
